(19) United States
(12) Patent Application Publication
(10) Pub. No.: US 2003/0220246 A1
(43) Pub. Date: Nov. 27, 2003

(54) Novel FGF homolog zFGF11

(76) Inventors: Darrell C. Conklin, Seattle, WA (US); Zhi Chen, Seattle, WA (US)

Correspondence Address: Deborah A. Sawislak, ZymoGenetics, Inc., Patent Department, 1201 Eastlake Avenue East, Seattle, WA, 98102, US

(21) Appl. No.: 10/347,143

(22) Filed: Jan. 17, 2003

Related U.S. Application Data
(62) Division of application No. 09/755,695, filed on Jan. 5, 2001, now abandoned.
(60) Provisional application No. 60/174,526, filed on Jan. 5, 2000.

(51) Int. Cl. A61K 38/18; C07H 21/04; C12P 21/02; C12N 5/06; C07K 14/50

(52) U.S. Cl. 514/12; 530/399; 536/23.5; 435/69.1; 435/320.1; 435/325

(57) ABSTRACT

The present invention relates to polynucleotide and polypeptide molecules for zFGF11 a novel member of the FGF family, which is most closely related to FGF 19 at the amino acid sequence level. The present invention also includes antibodies to the ZFGF11 polypeptides, and methods of using the polynucleotides and polypeptides.

### REFERENCE TO RELATED APPLICATIONS

This application is a divisional of U.S. application Ser. No. 09/755,695, filed on Jan. 5, 2001, which is related to U.S. Provisional Application 60/174,526, filed on Jan. 5, 2000, for which claims of benefit are made under 35 U.S.C. §120 and 35 U.S.C. §119(e)(1), and is incorporated herein in its entirety.

### BACKGROUND OF THE INVENTION

The fibroblast growth factor (FGF) family consists of at least eighteen distinct members (Basilico et al., Adv. Cancer Res. 59:115-165, 1992 and Fernig et al., Prog. Growth Factor Res. 5(4):353-377, 1994) which generally act as mitogens for a broad spectrum of cell types. For example, basic FGF (also known as FGF-2) is mitogenic in vitro for endothelial cells, vascular smooth muscle cells, fibroblasts, and generally for cells of mesoderm or neuroectoderm origin, including cardiac and skeletal myocytes (Gospodarowicz et al., J. Cell. Biol. 70:395-405, 1976; Gospodarowicz et al., J. Cell. Biol. 89:568-578, 1981 and Kardami, J. Mol. Cell. Biochem. 92:124-134, 1990). In vivo, bFGF has been shown to play a role in avian cardiac development (Sugi et al., Dev. Biol. 168:567-574, 1995 and Mima et al., Proc. Nat'l. Acad. Sci. 92:467-471, 1995), and to induce coronary collateral development in dogs (Lazarous et al., Circulation 94:1074-1082, 1996). In addition, non-mitogenic activities have been demonstrated for various members of the FGF family. Non-proliferative activities associated with acidic and/or basic FGF include: increased endothelial release of tissue plasminogen activator, stimulation of extracellular matrix synthesis, chemotaxis for endothelial cells, induced expression of fetal contractile genes in cardiomyocytes (Parker et al., J. Clin. Invest. 85:507-514, 1990), and enhanced pituitary hormonal responsiveness (Baird et al., J. Cellular Physiol. 5:101-106, 1987.)

Several members of the FGF family do not have a signal sequence (aFGF, bFGF and possibly FGF-9) and thus would not be expected to be secreted in a classical fashion. In addition, several of the FGF family members have the ability to migrate to the cell nucleus (Friesel et al., FASEB 9:919-925, 1995). All the members of the FGF family bind heparin based on structural similarities. Structural homology crosses species, suggesting a conservation of their structure/function relationship (Ornitz et al., J. Biol. Chem. 271(25):15292-15297, 1996.)

There are four known extracellular FGF receptors (FGFRs), and they are all tyrosine kinases. In general, the FGF family members bind to all of the known FGFRs, however, specific FGFs bind to specific receptors with higher degrees of affinity. Another means for specificity within the FGF family is the spatial and temporal expression of the ligands and their receptors during embryogenesis. Evidence suggests that the FGFs most likely act only in autocrine and/or paracrine manner, due to their heparin binding affinity, which limits their diffusion from the site of release (Flaumenhaft et al., J. Cell. Biol. 111(4):1651-1659, 1990.) Basic FGF lacks a signal sequence, and is therefore restricted to paracrine or autocrine modes of action. It has been postulated that basic FGF is stored intracellularly and released upon tissue damage. Basic FGF has been shown to have two receptor binding regions that are distinct from the heparin binding site (Abraham et al., EMBO J. 5(10):2523-2528, 1986.)

Within the FGF family, FGF-19 has been shown to have specificity not seen with other members of the family. It is generally believed that specificity is based on expression of the receptor, not the ligand, and therefore, it is believed that FGF-19 binds the FGFr4 receptor. The result of the specificity on the FGF-19/FGFr4 complex is that, unlike other FGFs, FGF-19 is not mitogenic for certain fibroblast cell lines (Botstein et al. WO 99/27100).

Members of the FGF family have been shown to play important roles developmentally and in adult tissue. The activities of the family members appear to be promiscuous in some tissues and have tissue-specificity in other cases. The present invention provides a novel member of the FGF family and the uses for these polynucleotides and polypeptides should be apparent to those skilled in the art from the teachings herein.

### BRIEF DESCRIPTION OF THE DRAWINGS

FIG. 1 is a alignment of human FGF-19 (SEQ ID NO: 4) and zFGF11 (SEQ ID NO: 2).

FIG. 2 is a Hopp/Woods hydrophilicity profile of the zFGF11 protein sequence shown in SEQ ID NO:2. The profile is based on a sliding six-residue window. Buried G, S, and T residues and exposed H, Y, and W residues were ignored. These residues are indicated in the figure by lower case letters.

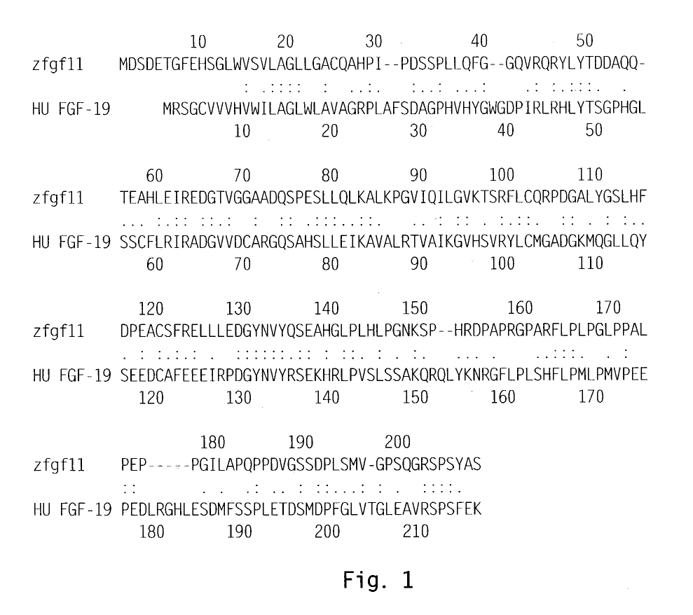

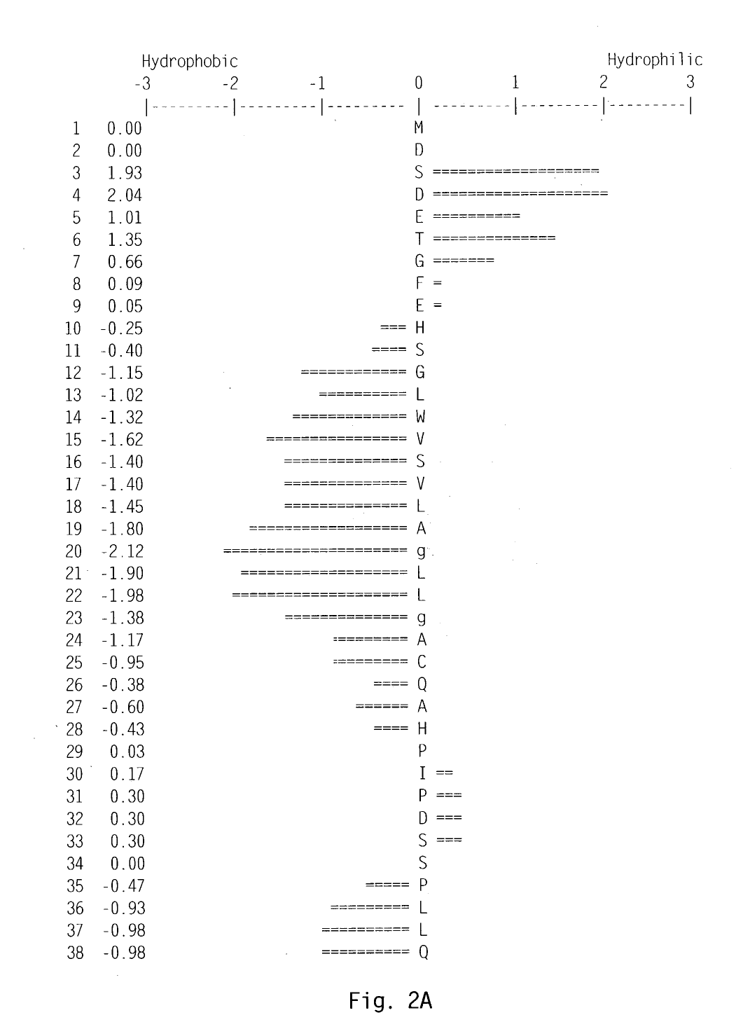

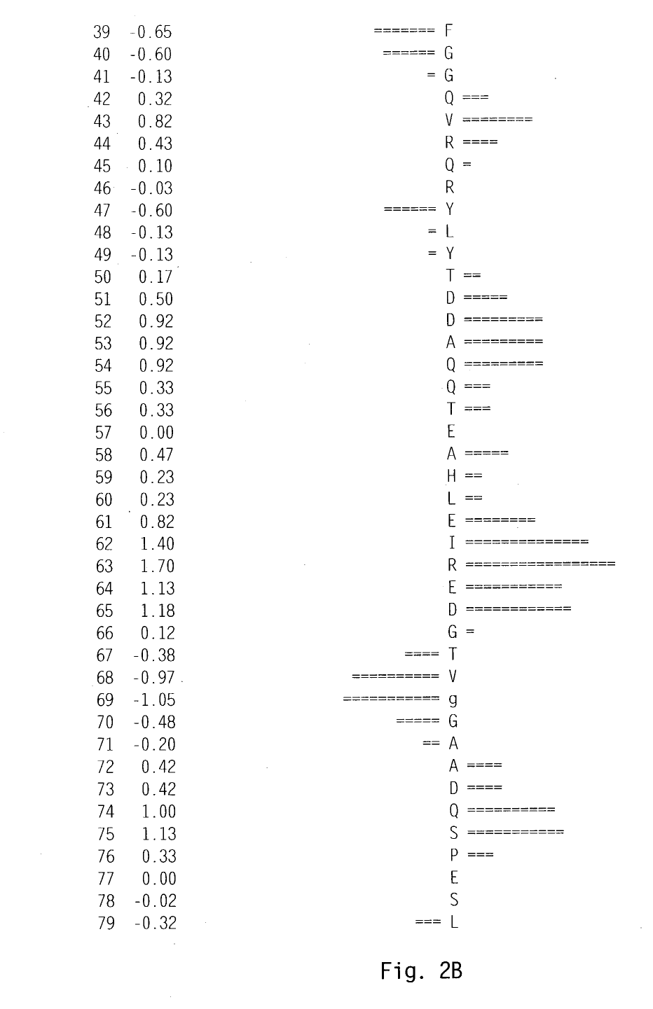

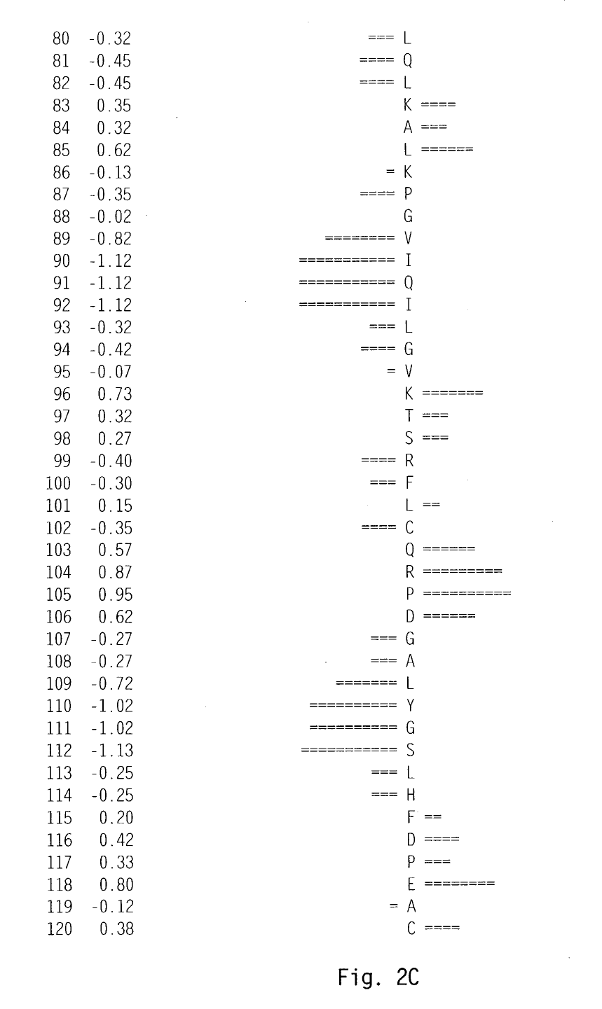

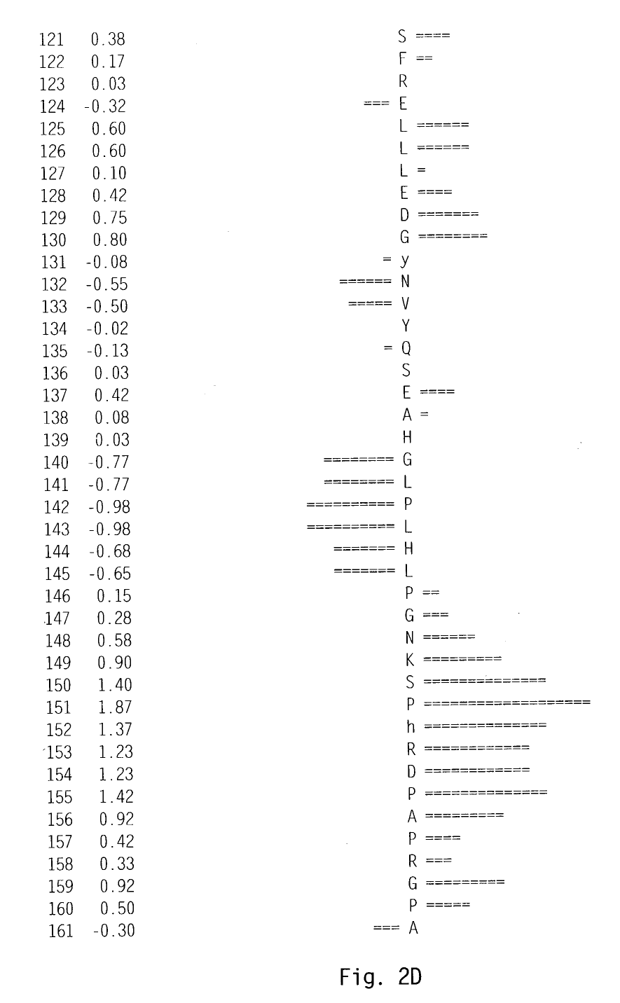

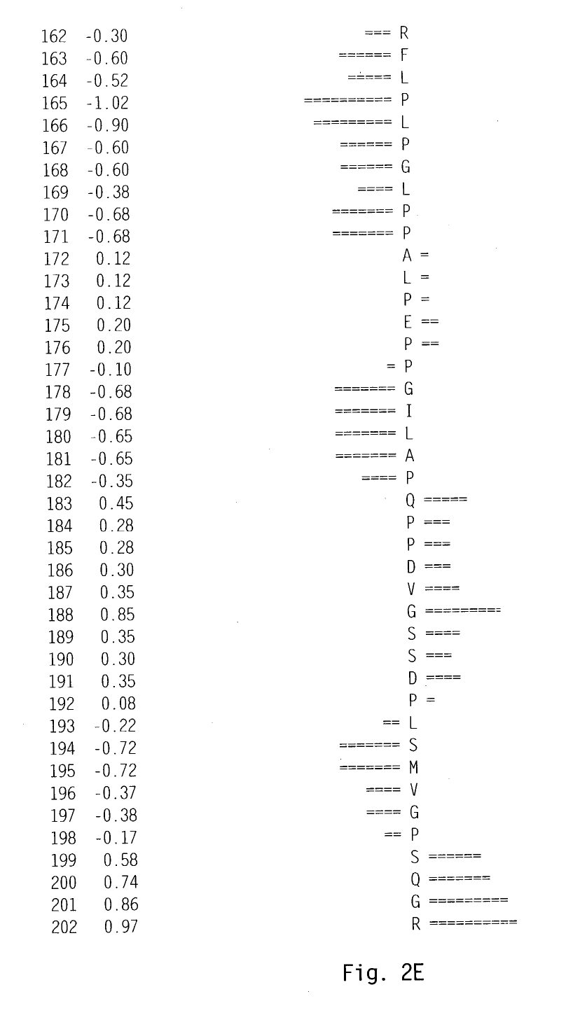

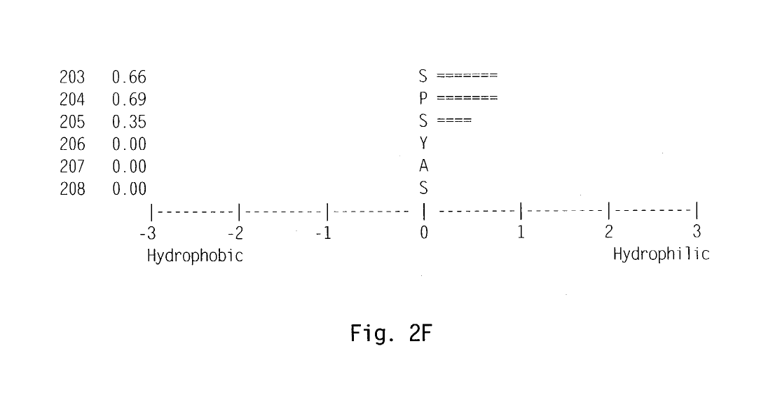

### DETAILED DESCRIPTION OF THE INVENTION

Prior to setting forth the invention in detail, it may be helpful to the understanding thereof to define the following terms:

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “complements of a polynucleotide molecule” is a polynucleotide molecule having a complementary base sequence and reverse orientation as compared to a reference sequence. For example, the sequence 5′ ATGCACGGG 3′ is complementary to 5′ CCCGTGCAT 3′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation.

The term “ortholog” denotes a polypeptide or protein obtained from one species that is the functional counterpart of a polypeptide or protein from a different species. Sequence differences among orthologs are the result of speciation.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel DNA sequence that encodes a fibroblast growth factor (FGF) homolog polypeptide having approximately 35% homology to FGF-19 (Nishimura et al., Biochem. Biophy. Acta 1444:148-151, 1999). The FGF homolog polypeptide has been designated zFGF11.

The novel zFGF11 polypeptides of the present invention contain a motif known to occur in all known members of the FGF family, and is unique to these proteins. The zFGF11 homolog polypeptide encoded by DNA contains a motif of the formula: CXFXE, wherein X is any amino acid and X{} is the number of X amino acids greater than one (SEQ ID NO: 5). This motif is highly conserved in all members of the FGF family, and a consensus amino acid sequence of the domain includes, for example, human myocyte-activating factor (FGF-10; HSU76381, GENBANK identifier,), human fibroblast growth factor homologous factor 4 (FHF-4; Smallwood et al., 1996, ibid.), human fibroblast growth factor homologous factor 3 (FHF-3; Smallwood et al., 1996, ibid.), human FGF-4 (Basilico et al., Adv. Cancer Res. 59:115-165,1992), human FGF-6 (Basilico et al., 1992, ibid.), human FGF-2 (basic; Basilico et al., 1992, ibid.), human FGF-1 (acidic; Basilico et al., 1992, ibid.), human keratinocyte growth factor precursor (FGF-7; Basilico et al., 1992, ibid.), human FGF-5 (Basilico et al., 1992, ibid.), human FGF-9 (Miyamoto et al., Mol. Cell. Biol. 13:4251-4259, 1993), human FGF-3 (Basilico et al., 1992, ibid.), human FGF-16 (Miyake et al., Biochem. Biophys. Res. Commun. 243(1):148-152, 1998) and human FGF-12 (Kok et al., Biochem. Biophys. Res. Commun. 255(3):717-721, 1999).

The DNA sequence as shown in SEQ ID NO. 1, has a genomic sequence that is common to many members of the FGF family, which comprises three exons separated by two introns. The deduced amino acid sequence is shown in SEQ ID NO: 2. Analysis of the DNA encoding a zFGF11 polypeptide (SEQ ID NO: 1) revealed three exons when spliced together formed an open reading frame encoding 208 amino acids (SEQ ID NO: 2) comprising a mature polypeptide of 181 amino acids (residue 28 to residue 208 of SEQ ID NO: 2) with a secretory signal sequence of 27 amino acids (residue 1 to 27 of SEQ ID NO: 2). Multiple alignment of zFGF11 with other known FGFs revealed a block of high percent identity corresponding to amino acid residue 89 to 138 of SEQ ID NO: 2. The FGF family motif, as shown in SEQ ID NO: 5, corresponds to amino acid residues 120 (Cys) to 124 (Glu) of SEQ ID NO: 2. Several of the members of the FGF family do not have signal sequences.

Members of the FGF family are characterized by heparin binding domains. A putative heparin-binding domain for zFGF11 has been identified in the region of amino acid residue 44 to amino acid residue 46 of SEQ ID NO: 2. It is postulated that receptor-mediated signaling is initiated upon binding of FGF ligand complexed with cell-surface heparin sulfate proteoglycans.

Based on homology alignments with FGF-1 and FGF-2 crystal structures (Eriksson et al., Prot. Sci. 2:1274, 1993), secondary structure predictions for beta strand structure of zFGF11 includes the following regions of amino acid residues: strand 2-58 (Ala)-64 (Glu); strand 3-66 (Gly)-72 (Ala); strand 4-79 (Leu)-84 (Ala); strand 5-88 (Gly)-84 (Val); strand 6-99 (Arg)-105 (Pro); strand 7-106 (Asp)-112 (Ser); strand 8-120 (Cys)-137 (Leu); and strand 9-130 (Gly)-147 (Glu), as shown in SEQ ID NO: 2. Amino acids critical for zFGF11 binding to receptors can be identified by site-directed mutagenesis of the entire zFGF11 polypeptide. More specifically, they can be identified using site-directed mutagenesis of amino acids in the zFGF11 polypeptide which correspond to amino acid residues in acidic FGF (FGF1) and basic FGF (FGF2) which have been identified as critical for binding to their respective receptors (Blaber et al., Biochem. 35:2086-2094, 1996). In zFGF11 hydrophobic residues buried within the core of the protein will be relatively intolerant of substitution, particularly polar or charged residues. Residues critical to the beta-trefoil fold of the zFGF11 include residues 60 (Leu), 68 (Val), 80 (Leu), 82 (Leu), 90 (Ile), 92 (Ile), 101 (Leu), 109 (Leu), 122 (Phe), and 134 (Tyr). One skilled in the art will recognize that other members, in whole or in part, of the FGF family may have structural or biochemical similarities to zFGF11. Therefore, amino acid residues from another FGF family member can be used for substitutions at corresponding positions in zFGF11 given the limitations disclosed herein.

Those skilled in the art will recognize that predicted domain boundaries are somewhat imprecise and may vary by up to ±3 amino acid residues.

Polypeptides of the present invention comprise at least 6, at least 9, or at least 15 contiguous amino acid residues of SEQ ID NO:2. Within certain embodiments of the invention, the polypeptides comprise 20, 30, 40, 50, 100, or more contiguous residues of SEQ ID NO:2, up to the entire predicted mature polypeptide (residues 28 to 208 of SEQ ID NO:2) or the primary translation product (residues 1 to 208 of SEQ ID NO:2). As disclosed in more detail below, these polypeptides can further comprise additional, non-zFGF 11, polypeptide sequence(s).

Within the polypeptides of the present invention are polypeptides that comprise an epitope-bearing portion of a protein as shown in SEQ ID NO:2. An “epitope” is a region of a protein to which an antibody can bind. See, for example, Geysen et al., Proc. Natl. Acad. Sci. USA 81:3998-4002, 1984. Epitopes can be linear or conformational, the latter being composed of discontinuous regions of the protein that form an epitope upon folding of the protein. Linear epitopes are generally at least 6 amino acid residues in length. Relatively short synthetic peptides that mimic part of a protein sequence are routinely capable of eliciting an antiserum that reacts with the partially mimicked protein. See, Sutcliffe et al., Science 219:660-666; 1983. Antibodies that recognize short, linear epitopes are particularly useful in analytic and diagnostic applications that employ denatured protein, such as Western blotting (Tobin, Proc. Natl. Acad. Sci. USA 76:4350-4356, 1979), or in the analysis of fixed cells or tissue samples. Antibodies to linear epitopes are also useful for detecting fragments of zFGF11, such as might occur in body fluids or cell culture media.

Antigenic, epitope-bearing polypeptides of the present invention are useful for raising antibodies, including monoclonal antibodies, that specifically bind to a zFGF11 protein. Antigenic, epitope-bearing polypeptides contain a sequence of at least six, preferably at least nine, more preferably from 15 to about 30 contiguous amino acid residues of a zFGF11 protein (e.g., SEQ ID NO:2). Polypeptides comprising a larger portion of a zFGF11 protein, i.e. from 30 to 50 residues up to the entire sequence, are included. It is preferred that the amino acid sequence of the epitope-bearing polypeptide is selected to provide substantial solubility in aqueous solvents, that is the sequence includes relatively hydrophilic residues, and hydrophobic residues are substantially avoided. Specific, useful polypeptides in this regard include those comprising residues 2-7, 1-6, 149-154, 61-66, and 60-65 of SEQ ID NO:2.

Polypeptides of the present invention can be prepared with one or more amino acid substitutions, deletions or additions as compared to SEQ ID NO:2. These changes are preferably of a minor nature, that is conservative amino acid substitutions and other changes that do not significantly affect the folding or activity of the protein or polypeptide as described herein. These changes include amino- or carboxyl-terminal extensions, such as an amino-terminal methionine residue, an amino or carboxyl-terminal cysteine residue to facilitate subsequent linking to maleimide-activated keyhole limpet hemocyanin, a small linker peptide of up to about 20-25 residues, or an extension that facilitates purification (an affinity tag) as disclosed above. Two or more affinity tags may be used in combination. Polypeptides comprising affinity tags can further comprise a polypeptide linker and/or a proteolytic cleavage site between the zFGF11 polypeptide and the affinity tag. Preferred cleavage sites include thrombin cleavage sites and factor Xa cleavage sites.

The present invention further provides a variety of other polypeptide fusions. For example, a zFGF11 polypeptide can be prepared as a fusion to a dimerizing protein as disclosed in U.S. Pat. Nos. 5,155,027 and 5,567,584. Preferred dimerizing proteins in this regard include immunoglobulin constant region domains. Immunoglobulin-zFGF11 polypeptide fusions can be expressed in genetically engineered cells to produce a variety of multimeric zFGF11 analogs. In addition, a zFGF11 polypeptide can be joined to another bioactive molecule, such as a cytokine, to provide a multi-functional molecule. One or more helices of a zFGF11 polypeptide can be joined to another cytokine to enhance or otherwise modify its biological properties. Auxiliary domains can be fused to zFGF11 polypeptides to target them to specific cells, tissues, or macromolecules (e.g., collagen). For example, a zFGF11 polypeptide or protein can be targeted to a predetermined cell type by fusing a zFGF11 polypeptide to a ligand that specifically binds to a receptor on the surface of the target cell. In this way, polypeptides and proteins can be targeted for therapeutic or diagnostic purposes. A zFGF 11 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

Polypeptide fusions of the present invention will generally contain not more than about 1,500 amino acid residues, preferably not more than about 1,200 residues, more preferably not more than about 1,000 residues, and will in many cases be considerably smaller. For example, a zFGF11 polypeptide of 181 residues (residues 28-208 of SEQ ID NO:2) can be fused to E. coli β-galactosidase (1,021 residues; see Casadaban et al., J. Bacteriol. 143:971-980, 1980), a 10-residue spacer, and a 4-residue factor Xa cleavage site. In a second example, residues 28-208 SEQ ID NO:2 can be fused to maltose binding protein (approximately 370 residues), a 4-residue cleavage site, and a 6-residue polyhistidine tag.

As disclosed above, the polypeptides of the present invention comprise at least 6 contiguous residues of SEQ ID NO:2. These polypeptides may further comprise additional residues as shown in SEQ ID NO:2, a variant of SEQ ID NO:2, or another protein as disclosed herein. When variants of SEQ ID NO:2 are employed, the resulting polypeptide will be at least 80% to 90% identical or in other embodiments, at least 95%, 96%, 97%, 98%, or 99% identical to the corresponding region of SEQ ID NO:2. Percent sequence identity is determined by conventional methods. See, for example, Altschul et al., Bull. Math. Bio. 48:603-616, 1986, and Henikoff and Henikoff, Proc. Natl. Acad. Sci. USA 89:10915-10919, 1992. Briefly, two amino acid sequences are aligned to optimize the alignment scores using a gap opening penalty of 10, a gap extension penalty of 1, and the “BLOSUM62” scoring matrix of Henikoff and Henikoff (ibid.) as shown in Table 1 (amino acids are indicated by the standard one-letter codes). The percent identity is then calculated as:

$$\frac{Total number of identical matches}{\begin{matrix}\begin{matrix}[ length of the longer sequence plus the \\ number of gaps introduced into the longer\end{matrix} \\ sequence in order to align the two sequences ]\end{matrix}} \times 100$$

TABLE 1

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The level of identity between amino acid sequences can be determined using the “FASTA” similarity search algorithm disclosed by Pearson and Lipman (Proc. Natl. Acad. Sci. USA 85:2444, 1988) and by Pearson (Meth. Enzymol. 183:63, 1990). Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, 1990 (ibid.).

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

The present invention includes polypeptides having one or more conservative amino acid changes as compared with the amino acid sequence of SEQ ID NO:2. The BLOSUM62 matrix (Table 1) is an amino acid substitution matrix derived from about 2,000 local multiple alignments of protein sequence segments, representing highly conserved regions of more than 500 groups of related proteins (Henikoff and Henikoff, ibid.). Thus, the BLOSUM62 substitution frequencies can be used to define conservative amino acid substitutions that may be introduced into the amino acid sequences of the present invention. As used herein, the term “conservative amino acid substitution” refers to a substitution represented by a BLOSUM62 value of greater than −1. For example, an amino acid substitution is conservative if the substitution is characterized by a BLOSUM62 value of 0, 1, 2, or 3. Preferred conservative amino acid substitutions are characterized by a BLOSUM62 value of at least one 1 (e.g., 1, 2 or 3), while more preferred conservative amino acid substitutions are characterized by a BLOSUM62 value of at least 2 (e.g., 2 or 3).

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-809, 1993; and Chung et al., Proc. Natl. Acad. Sci. USA 90:10145-10149, 1993). In a second method, translation is carried out in Xenopus oocytes by microinjection of mutated mRNA and chemically aminoacylated suppressor tRNAs (Turcatti et al., J. Biol. Chem. 271:19991-19998, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-7476, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

Amino acid sequence changes are made in zFGF11 polypeptides so as to minimize disruption of higher order structure essential to biological activity as disclosed previously. Amino acid residues that are within regions or domains that are critical to maintaining structural integrity can be determined. Within these regions one can identify specific residues that will be more or less tolerant of change and maintain the overall tertiary structure of the molecule. Methods for analyzing sequence structure include, but are not limited to, alignment of multiple sequences with high amino acid or nucleotide identity, secondary structure propensities, binary patterns, complementary packing, and buried polar interactions (Barton, Current Opin. Struct. Biol. 5:372-376, 1995 and Cordes et al., Current Opin. Struct. Biol. 6:3-10, 1996). In general, determination of structure will be accompanied by evaluation of activity of modified molecules. For example, changes in amino acid residues will be made so as not to disrupt the beta-trefoil fold structure of the protein family. The effects of amino acid sequence changes can be predicted by, for example, computer modeling using available software (e.g., the Insight II® viewer and homology modeling tools; MSI, San Diego, Calif.) or determined by analysis of crystal structure (see, e.g., Lapthorn et al, Nature 369:455-461, 1994; Lapthorn et al., Nat. Struct. Biol. 2:266-268, 1995). Protein folding can be measured by circular dichroism (CD). Measuring and comparing the CD spectra generated by a modified molecule and standard molecule are routine in the art (Johnson, Proteins 7:205-214, 1990). Crystallography is another well known and accepted method for analyzing folding and structure. Nuclear magnetic resonance (NMR), digestive peptide mapping and epitope mapping are other known methods for analyzing folding and structural similarities between proteins and polypeptides (Schaanan et al., Science 257:961-964, 1992). Mass spectrometry and chemical modification using reduction and alkylation can be used to identify cysteine residues that are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). Alterations in disulfide bonding will be expected to affect protein folding. These techniques can be employed individually or in combination to analyze and compare the structural features that affect folding of a variant protein or polypeptide to a standard molecule to determine whether such modifications would be significant.

Essential amino acids in the polypeptides of the present invention can be identified experimentally according to procedures known in the art, such as site-directed mutagenesis or alanine-scanning mutagenesis (Cunningham and Wells, Science 244, 1081-1085, 1989; Bass et al., Proc. Natl. Acad. Sci. USA 88:4498-4502, 1991). In the latter technique, single alanine mutations are introduced at every residue in the molecule, and the resultant mutant molecules are tested for biological activity as disclosed below to identify amino acid residues that are critical to the activity of the molecule.

Multiple amino acid substitutions can be made and tested using known methods of mutagenesis and screening, such as those disclosed by Reidhaar-Olson and Sauer (Science 241:53-57, 1988) or Bowie and Sauer (Proc. Natl. Acad. Sci. USA 86:2152-2156, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-10837, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed zFGF11 DNA and polypeptide sequences can be generated through DNA shuffling as disclosed by Stemmer, Nature 370:389-391, 1994 and Stemmer, Proc. Natl. Acad. Sci. USA 91:10747-10751, 1994. Briefly, variant genes are generated by in vitro homologous recombination by random fragmentation of a parent gene followed by reassembly using PCR, resulting in randomly introduced point mutations. This technique can be modified by using a family of parent genes, such as allelic variants or genes from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

In many cases, the structure of the final polypeptide product will result from processing of the nascent polypeptide chain by the host cell, thus the final sequence of a zFGF11 polypeptide produced by a host cell will not always correspond to the full sequence encoded by the expressed polynucleotide. For example, expressing the complete zFGF11 sequence in a cultured mammalian cell is expected to result in removal of at least the secretory peptide, while the same polypeptide produced in a prokaryotic host would not be expected to be cleaved. Differential processing of individual chains may result in heterogeneity of expressed polypeptides.

SEQ ID NO: 3 is a degenerate polynucleotide sequence that encompasses all polynucleotides that could encode the zFGF11 polypeptide of SEQ ID NO: 2 (amino acids 1 or 28 to 208). Thus, zFGF11 polypeptide-encoding polynucleotides ranging from nucleotide 1 or 82 to nucleotide 624 of SEQ ID NO: 3 are contemplated by the present invention. Also contemplated by the present invention are fragments and fusions as described above with respect to SEQ ID NO: 1, which are formed from analogous regions of SEQ ID NO: 3, wherein nucleotides 1 or 82 to 624 of SEQ ID NO: 3 correspond to nucleotides 150 or 231 to 776 of SEQ ID NO: 1, for the encoding a mature zFGF11 molecule.

The symbols in SEQ ID NO: 3 are summarized in Table 2 below.

TABLE 2

| Nucleotide | Resolutions | Complement | Resolutions |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| C\|G | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO: 3, encompassing all possible codons for a given amino acid, are set forth in Table 3.

TABLE 3

| | | | |
|---|---|---|---|
| Amino | | | Degenerate |
| Acid | Letter | Codons | Codon |

| | | | |
|---|---|---|---|
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TPA TAG TGA | TRR |
| Asn\|Asp | B | | RAY |
| Glu\|Gln | Z | | SAR |
| Any | X | | NNN |
| Gap | — | | |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may have some variant amino acids, but one of ordinary skill in the art can easily identify such variant sequences by reference to the amino acid sequence of SEQ ID NO: 2. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid Threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO: 3 serves as a template for optimizing expression of polynucleotides in various cell types and species commonly used in the art and disclosed herein. Sequences containing preferential codons can be tested and optimized for expression in various species, and tested for functionality as disclosed herein.

The highly conserved amino acids in zFGF11 can be used as a tool to identify new family members. To identify new family members in EST databases, the conserved CXFXE motif (SEQ ID NO: 5) can be used. In another method using polynucleotide probes and hybridization methods, RNA obtained from a variety of tissue sources can be used to generate cDNA libraries and probe these libraries for new family members. In particular, reverse transcription-polymerase chain reaction (RT-PCR) can be used to amplify sequences encoding highly degenerate DNA primers designed from the sequences corresponding to amino acid residue 113 (Cys) to amino acid residue 117 (His) of SEQ ID NO: 2.

The zFGF11 gene has been derived chromosome 19 (Genome Catalog, Oakridge National Laboratory, Oakridge, Tenn.). Thus, the present invention provides methods for using zFGF11 polynucleotides and polypeptides to identify chromosomal disorders associated with abnormal expression of the zFGF11 protein. Detectable chromosomal mutations at the zFGF11 gene locus include, but are not limited to, aneuploidy, gene copy number changes, insertions, deletions, translocations, restriction site changes and rearrangements. Such aberrations can be identified by employing molecular genetic techniques, such as restriction fragment length polymorphism (RFLP) analysis, short tandem repeat (STR) analysis employing PCR techniques, and other genetic linkage analysis techniques known in the art (Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987; A. J. Marian, Chest 108:255-65, 1995). Analyses of DNA samples can detect deletions and insertions by changes in size in amplified DNA products by comparing a sample DNA to a normal zFGF11 DNA standard. Mismatches in duplex DNA can be detected by RNase digestion or differences in melting temperature. Other methods for detecting differences in sequences include changes in electrophoretic motility, Southern analysis, and direct DNA sequencing. Recently, techniques for accessing genetic information with high-density arrays have been available (Chee et al., Science 274:610-614, 1996), and can analyze large fragments of genomic DNA with high resolution.

Analysis of chromosomal DNA using the zFGF11 polynucleotide sequence is useful for correlating disease with mutations localized to the chromosome where the zFGF11 gene resides. Studies of the DNA sequences, cDNA and/or genomic DNA, of individuals presenting disease that correlates with a mutation in the sequence of the zFGF11 gene, wherein such mutation is not present in normal individuals, can provide strong evidence for the mutation as causative factor of the disease. In one embodiment, the methods of the present invention provide a method of detecting a zFGF11 chromosomal abnormality in sample from an individual comprising: (a) obtaining a zFGF11 RNA from the sample; (b) generating zFGF11 cDNA by polymerase chain reaction; and (c) comparing the nucleic acid sequence of the zFGF11 cDNA to the nucleic acid sequence as shown in SEQ ID NO: 1. In further embodiments, the difference between the sequence of the zFGF11 cDNA or zFGF11 gene in the sample and the zFGF11 sequence as shown in SEQ ID NO: 1 is indicative of a zFGF11 chromosomal mutation. In other embodiments, introns, splice acceptor or splice donor abnormalities can be detected by comparison of genomic sequences from a patient to a standard genomic sequence.

The protein truncation test is also useful for detecting the inactivation of a gene in which translation-terminating mutations produce only portions of the encoded protein (see, for example, Stoppa-Lyonnet et al., Blood 91:3920 (1998)). According to this approach, RNA is isolated from a biological sample, and used to synthesize cDNA. PCR is then used to amplify the ZFGF11 target sequence and to introduce an RNA polymerase promoter, a translation initiation sequence, and an in-frame ATG triplet. PCR products are transcribed using an RNA polymerase, and the transcripts are translated in vitro with a T7-coupled reticulocyte lysate system. The translation products are then fractionated by SDS-PAGE to determine the lengths of the translation products. The protein truncation test is described, for example, by Dracopoli et al. (eds.), Current Protocols in Human Genetics, pages 9.11.1-9.11.18 (John Wiley & Sons 1998).

The present invention also contemplates kits for performing a diagnostic assay for ZFGF11 gene expression or to analyze the ZFGF11 locus of a subject. Such kits comprise nucleic acid probes, such as double-stranded nucleic acid molecules comprising the nucleotide sequence of SEQ ID NOS:1 or 9, or a fragment thereof, as well as single-stranded nucleic acid molecules having the complement of the nucleotide sequence of SEQ ID NOS:1 or 9, or a fragment thereof. Probe molecules may be DNA, RNA, oligonucleotides, and the like. Kits may comprise nucleic acid primers for performing PCR.

Such a kit can contain all the necessary elements to perform a nucleic acid diagnostic assay described above. A kit will comprise at least one container comprising a ZFGF11 probe or primer. The kit may also comprise a second container comprising one or more reagents capable of indicating the presence of ZFGF11 sequences. Examples of such indicator reagents include detectable labels such as radioactive labels, fluorochromes, chemiluminescent agents, and the like. A kit may also comprise a means for conveying to the user that the ZFGF11 probes and primers are used to detect ZFGF11 gene expression. For example, written instructions may state that the enclosed nucleic acid molecules can be used to detect either a nucleic acid molecule that encodes ZFGF11, or a nucleic acid molecule having a nucleotide sequence that is complementary to a ZFGF11-encoding nucleotide sequence, or to analyze chromosomal sequences associated with the ZFGF11 locus. The written material can be applied directly to a container, or the written material can be provided in the form of a packaging insert.

Within preferred embodiments of the invention, the isolated nucleic acid molecules can hybridize under stringent conditions to nucleic acid molecules having at least a portion of the nucleotide sequence of SEQ ID NOs:1 or 3 or to nucleic acid molecules having a nucleotide sequence complementary to those sequences. A pair of nucleic acid molecules, such as DNA-DNA, RNA-RNA and DNA-RNA, can hybridize if the nucleotide sequences have some degree of complementarity. Hybrids can tolerate mismatched base pairs in the double helix, but the stability of the hybrid is influenced by the degree of mismatch. The Tₘ of the mismatched hybrid decreases by 1° C. for every 1-1.5% base pair mismatch. Varying the stringency of the hybridization conditions allows control over the degree of mismatch that will be present in the hybrid. The degree of stringency increases as the hybridization temperature increases and the ionic strength of the hybridization buffer decreases. Stringent hybridization conditions encompass temperatures of about 5-25° C. below the Tₘ of the hybrid and a hybridization buffer having up to 1 M Na⁺. Higher degrees of stringency at lower temperatures can be achieved with the addition of formamide which reduces the Tₘ of the hybrid about 1° C. for each 1% formamide in the buffer solution. Generally, such stringent conditions include temperatures of 20-70° C. and a hybridization buffer containing up to 6×SSC and 0-50% formamide. A higher degree of stringency can be achieved at temperatures of from 40-70° C. with a hybridization buffer having up to 4×SSC and from 0-50% formamide. Highly stringent conditions typically encompass temperatures of 42-70° C. with a hybridization buffer having up to 1×SSC and 0-50% formamide. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes.

The above conditions are meant to serve as a guide and it is well within the abilities of one skilled in the art to adapt these conditions for use with a particular polypeptide hybrid. The Tₘ for a specific target sequence is the temperature (under defined conditions) at which 50% of the target sequence will hybridize to a perfectly matched probe sequence. Those conditions which influence the Tₘ include, the size and base pair content of the polynucleotide probe, the ionic strength of the hybridization solution, and the presence of destabilizing agents in the hybridization solution. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software, such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ. For smaller probes, <50 base pairs, hybridization is typically carried out at the Tₘ or 5-10° C. below. This allows for the maximum rate of hybridization for DNA-DNA and DNA-RNA hybrids.

The length of the polynucleotide sequence influences the rate and stability of hybrid formation. Smaller probe sequences, <50 base pairs, reach equilibrium with complementary sequences rapidly, but may form less stable hybrids. Incubation times of anywhere from minutes to hours can be used to achieve hybrid formation. Longer probe sequences come to equilibrium more slowly, but form more stable complexes even at lower temperatures. Incubations are allowed to proceed overnight or longer. Generally, incubations are carried out for a period equal to three times the calculated Cot time. Cot time, the time it takes for the polynucleotide sequences to reassociate, can be calculated for a particular sequence by methods known in the art.

The base pair composition of polynucleotide sequence will effect the thermal stability of the hybrid complex, thereby influencing the choice of hybridization temperature and the ionic strength of the hybridization buffer. A-T pairs are less stable than G-C pairs in aqueous solutions containing sodium chloride. Therefore, the higher the G-C content, the more stable the hybrid. Even distribution of G and C residues within the sequence also contribute positively to hybrid stability. In addition, the base pair composition can be manipulated to alter the Tₘ of a given sequence. For example, 5-methyldeoxycytidine can be substituted for deoxycytidine and 5-bromodeoxuridine can be substituted for thymidine to increase the Tₘ, whereas 7-deazz-2′-deoxyguanosine can be substituted for guanosine to reduce dependence on Tₘ.

The ionic concentration of the hybridization buffer also affects the stability of the hybrid. Hybridization buffers generally contain blocking agents such as Denhardt's solution (Sigma Chemical Co., St. Louis, Mo.), denatured salmon sperm DNA, tRNA, milk powders (BLOTTO), heparin or SDS, and a Na⁺ source, such as SSC (1×SSC: 0.15 M sodium chloride, 15 mM sodium citrate) or SSPE (1×SSPE: 1.8 M NaCl, 10 mM NaH₂PO₄, 1 mM EDTA, pH 7.7). By decreasing the ionic concentration of the buffer, the stability of the hybrid is increased. Typically, hybridization buffers contain from between 10 mM-1 M Na⁺. The addition of destabilizing or denaturing agents such as formamide, tetralkylammonium salts, guanidinium cations or thiocyanate cations to the hybridization solution will alter the Tₘ of a hybrid. Typically, formamide is used at a concentration of up to 50% to allow incubations to be carried out at more convenient and lower temperatures. Formamide also acts to reduce non-specific background when, using RNA probes.

As previously noted, the isolated polynucleotides of the present invention include DNA and RNA. Methods for preparing DNA and RNA are well known in the art. In general, RNA is isolated from a tissue or cell that produces large amounts of zFGF11 RNA. Such tissues and cells are identified by Northern blotting (Thomas, Proc. Natl. Acad. Sci. USA 77:5201, 1980), and include pancreas and prostate. Total RNA can be prepared using guanidinium isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-1412, 1972). Complementary DNA (cDNA) is prepared from poly(A)⁺ RNA using known methods. Polynucleotides encoding zFGF11 polypeptides are then identified and isolated by, for example, hybridization or PCR.

A full-length clone encoding zFGF11 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to zFGF11, receptor fragments, or other specific binding partners.

The present invention further provides counterpart polypeptides and polynucleotides from other species (orthologs). Of particular interest are zFGF11 polypeptides from other mammalian species, including murine, rat, porcine, ovine, bovine, canine, feline, equine and other primate proteins.

Orthologs of the human proteins can be cloned using information and compositions provided by the present invention in combination with conventional cloning techniques. For example, a cDNA can be cloned using mRNA obtained from a tissue or cell type that expresses the protein. Suitable sources of mRNA can be identified by probing Northern blots with probes designed from the sequences disclosed herein. A library is then prepared from mRNA of a positive tissue or cell line. A zFGF11-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the sequences disclosed herein. Within an additional method, the cDNA library can be used to transform or transfect host cells, and expression of the cDNA of interest can be detected with an antibody to zFGF11. Similar techniques can also be applied to the isolation of genomic clones.

Those skilled in the art will recognize that the sequences disclosed in SEQ ID NO: 1 and SEQ ID NO: 2 represent a single allele of the human zFGF11 gene and polypeptide, respectively, and that allelic variation and alternative splicing are expected to occur. Allelic variants can be cloned by probing cDNA or genomic libraries from different individuals according to standard procedures. Allelic variants of the DNA sequence shown in SEQ ID NO: 1, including those containing silent mutations and those in which mutations result in amino acid sequence changes, are within the scope of the present invention, as are proteins which are allelic variants of SEQ ID NO: 2.

Mutagenesis methods as disclosed above can be combined with high-throughput, automated screening methods to detect activity of cloned, mutagenized polypeptides in host cells. Mutagenized DNA molecules that encode active polypeptides (e.g., cell proliferation) can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can identify and/or prepare a variety of polypeptides that are substantially homologous to residues 28 (His) to 208 (Ser) or residues 1 (Met) to 208 (Ser) of SEQ ID NO: 2, allelic variants thereof, or biologically active fragments thereof, and retain the proliferative properties of the wild-type protein. Such polypeptides may also include additional polypeptide segments as generally disclosed above.

The polypeptides of the present invention, including full-length proteins, fragments thereof and fusion proteins, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al. (eds.), Current Protocols in Molecular Biology, John Wiley and Sons, Inc., NY, 1987, which are incorporated herein by reference.

In general, a DNA sequence encoding a zFGF11 polypeptide of the present invention is operably linked to other genetic elements required for its expression, generally including a transcription promoter and terminator within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

To direct a zFGF11 polypeptide into the secretory pathway of a host cell, a secretory signal sequence (also known as a leader sequence, prepro sequence or pre sequence) is provided in the expression vector. The secretory signal sequence may be the native sequence, or a chimera comprising a signal sequence derived from another secreted protein (e.g., t-PA and α-pre-pro secretory leader) or synthesized de novo. The secretory signal sequence is joined to the zFGF11 DNA sequence in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Alternatively, the secretory signal sequence contained in the polypeptides of the present invention is used to direct other polypeptides into the secretory pathway. The present invention provides for such fusion polypeptides. A signal fusion polypeptide can be made wherein a secretory signal sequence derived from amino acid residues 1-27 of SEQ ID NO:2 is be operably linked to another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein. Such fusions may be used in vivo or in vitro to direct peptides through the secretory pathway publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. A second method of making recombinant zFGF11 baculovirus utilizes a transposon-based system described by Luckow (Luckow, V. A, et al., J Virol 67:4566-79, 1993). This system, which utilizes transfer vectors, is sold in the Bac-to-Bac™ kit (Life Technologies, Rockville, Md.). This system utilizes a transfer vector, pFastBac1™ (Life Technologies) containing a Tn7 transposon to move the DNA encoding the zFGF11 polypeptide into a baculovirus genome maintained in E. coli as a large plasmid called a “bacmid.” See, Hill-Perkins, M. S. and Possee, R. D., J Gen Virol 71:971-6, 1990; Bonning, B. C. et al., J Gen Virol 75:1551-6, 1994; and, Chazenbalk, G. D., and Rapoport, B., J Biol Chem 270:1543-9, 1995. In addition, transfer vectors can include an in-frame fusion with DNA encoding an epitope tag at the C- or N-terminus of the expressed zFGF11 polypeptide, for example, a Glu-Glu epitope tag (Grussenmeyer, T. et al., Proc. Natl. Acad. Sci. 82:7952-4, 1985). Using a technique known in the art, a transfer vector containing zFGF11 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zFGF11 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the zFGF11 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme 4,977,092) 132:3459-65, 1986 and Cregg, U.S. Pat. No. 4,882,279. Aspergillus cells may be utilized 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61 or DG44) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978)and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

It is preferred to purify the polypeptides of the present invention to ≧80% purity, more preferably to ≧90% purity, even more preferably ≧95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant zFGF11 polypeptides (or chimeric zFGF11 polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable anion exchange media include derivatized dextrans, agarose, cellulose, polyacrylamide, specialty silicas, and the like. PEI, DEAE, QAE and Q derivatives are preferred, with DEAE Fast-Flow Sepharose (Pharmacia, Piscataway, N.J.) being particularly preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can also be isolated by exploitation of their heparin binding properties. For a review, see, Burgess et al., Ann. Rev. of Biochem. 58:575-606, 1989. Members of the FGF family can be purified to apparent homogeneity by heparin-Sepharose affinity chromatography (Gospodarowicz et al., Proc. Natl. Acad. Sci. 81:6963-6967, 1984) and eluted using linear step gradients of NaCl (Ron et al., J. Biol. Chem. 268(4):2984-2988, 1993; Chromatography: Principles & Methods, pp. 77-80, Pharmacia LKB Biotechnology, Uppsala, Sweden, 1993; in “Immobilized Affinity Ligand Techniques”, Hermanson et al., eds., pp. 165-167, Academic Press, San Diego, 1992; Kjellen et al., Ann. Rev. Biochem.Ann. Rev. Biochem. 60:443-474, 1991; and Ke et al., Protein Expr. Purif. 3(6):497-507, 1992.)

Other purification methods include using immobilized metal ion adsorption (IMAC) chromatography to purify histidine-rich proteins. Briefly, a gel is first charged with divalent metal ions to form a chelate (E. Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39). Alternatively, a fusion of the polypeptide of interest and an affinity tag (e.g., polyhistidine, maltose-binding protein, an immunoglobulin domain) may be constructed to facilitate purification.

Protein refolding (and optionally reoxidation) procedures may be advantageously used. It is preferred to purify the protein to >80% purity, more preferably to >90% purity, even more preferably >95%, and particularly preferred is a pharmaceutically pure state, that is greater than 99.9% pure with respect to contaminating macromolecules, particularly other proteins and nucleic acids, and free of infectious and pyrogenic agents. Preferably, a purified protein is substantially free of other proteins, particularly other proteins of animal origin.

zFGF11 polypeptides or fragments thereof may also be prepared through chemical synthesis. zFGF11 polypeptides may be monomers or multimers; glycosylated or non-glycosylated; pegylated or non-pegylated; and may or may not include an initial methionine amino acid residue.

An in vivo approach for assaying proteins of the present invention involves viral delivery systems. Exemplary viruses for this purpose include adenovirus, herpesvirus, vaccinia virus and adeno-associated virus (AAV). Adenovirus, a double-stranded DNA virus, is currently the best studied gene transfer vector for delivery of heterologous nucleic acid (for a review, see T. C. Becker et al., Meth. Cell Biol. 43:161-89, 1994; and J. T. Douglas and D. T. Curiel, Science & Medicine 4:44-53, 1997). The adenovirus system offers several advantages: adenovirus can (i) accommodate relatively large DNA inserts; (ii) be grown to high-titer; (iii) infect a broad range of mammalian cell types; and (iv) be used with a large number of available vectors containing different promoters. Also, because adenoviruses are stable in the bloodstream, they can be administered by intravenous injection.

By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293 cells can be grown as adherent cells or in suspension culture at relatively high cell density to produce significant amounts of protein (see Garnier et al., Cytotechnol. 15:145-55, 1994). With either protocol, an expressed, secreted heterologous protein can be repeatedly isolated from the cell culture supernatant. Within the infected 293S cell production protocol, non-secreted proteins may also be effectively obtained.

The activity of molecules of the present invention can be measured using a variety of assays that, for example, measure neogenesis or hyperplasia (i.e., proliferation) of neuronal, prostatic or pancreatic cells based on the tissue specificity. Additional activities likely associated with the polypeptides of the present invention include proliferation of endothelial cells, fibroblasts, skeletal myocytes, epithelial cells and keratinocytes, directly or indirectly through other growth factors; action as a chemotaxic factor for endothelial cells, fibroblasts and/or phagocytic cells; osteogenic factor; and factor for expanding mesenchymal stem cell and precursor populations.

Proliferation can be measured using cultured cardiac cells or in vivo by administering molecules of the claimed invention to the appropriate animal model. Generally, proliferative effects are seen as an increase in cell number and therefore, may include inhibition of apoptosis, as well as mitogenesis. Cultured cells include fibroblasts, cardiac or skeletal myocytes, human umbilical vein endothelial cells from primary cultures. Established cell lines include: NIH 3T3 fibroblast (ATCC No. CRL-1658), CHH-1 chum heart cells (ATCC No. CRL-1680), H9c2 rat heart myoblasts (ATCC No. CRL-1446), Shionogi mammary carcinoma cells (Tanaka et al., Proc. Natl. Acad. Sci. 89:8928-8932, 1992) and LNCap.FGC adenocarcinoma cells (ATCC No. CRL-1740.) Assays measuring cell proliferation are well known in the art. For example, assays measuring proliferation include such assays as chemosensitivity to neutral red dye (Cavanaugh et al., Investigational New Drugs 8:347-354, 1990, incorporated herein by reference), incorporation of radiolabelled nucleotides (Cook et al., Analytical Biochem. 179:1-7, 1989, incorporated herein by reference), incorporation of 5-bromo-2′-deoxyuridine (BrdU) in the DNA of proliferating cells (Porstmann et al., J. Immunol. Methods 82:169-179, 1985, incorporated herein by reference), and use of tetrazolium salts (Mosmann, J. Immunol. Methods 65:55-63, 1983; Alley et al., Cancer Res. 48:589-601, 1988; Marshall et al., Growth Reg. 5:69-84, 1995; and Scudiero et al., Cancer Res. 48:4827-4833, 1988; all incorporated herein by reference).

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made. Progenitor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products and receptors. The stage of a cell population's differentiation is monitored by identification of markers present in the cell population. Myocytes, osteoblasts, adipocytes, chrondrocytes, fibroblasts and reticular cells are believed to originate from a common mesenchymal stem cell (Owen et al., Ciba Fdn. Symp. 136:42-46, 1988). Markers for mesenchymal stem cells have not been well identified (Owen et al., J. of Cell Sci. 87:731-738, 1987), so identification is usually made at the progenitor and mature cell stages. The novel polypeptides of the present invention are useful for studies to isolate mesenchymal stem cells and myocyte progenitor cells, both in vivo and ex vivo.

There is evidence to suggest that factors that stimulate specific cell types down a pathway towards terminal differentiation or dedifferentiation, affects the entire cell population originating from a common precursor or stem cell. Thus, the present invention includes stimulation, inhibition, or proliferation of myocytes, smooth muscle cells, osteoblasts, adipocytes, chondrocytes, neural tube-derived stem cells, neural crest stem cells, and neuronal progenitors, pancreatic cells, prostate-derived cells and endothelial cells. Molecules of the present invention may, while stimulating proliferation or differentiation of cardiac myocytes, inhibit proliferation or differentiation of adipocytes, by virtue of the affect on their common precursor/stem cells. Thus molecules of the present invention, have use in inhibiting chondrosarcomas, atherosclerosis, restenosis and obesity.

Based on data that a receptor for zFGF11 is found on osteoblasts, the molecules of the present invention will be used for stimulating proliferation of osteoblasts, in vitro and in vivo. Stimulation of osteoblasts, resulting in bone formation will be useful for treatment of bone defects, fractures, osteoporosis and other deficiencies in bone structure and formation.

It has been suggested that specificity in the FGF family is a determined by the cognate receptor expression because there is significant promiscuity among the ligand/receptor complexes. Therefore, identification of FGFR1 IIIc as a binding partner for zFGF11 provides further support for both identifying target tissues and biological functions of the zFGF11 molecule FGFR1 IIIc has been identified by Northern analysis as being highly expressed in epidermis, dermis, brain, kidney, skeletal muscle, heart and lung (Beer et al., J. Biol. Chem. 275:16091-16097, 2000). Using in situ hybridization, the FGFR1 IIIc has been identified in osteoblasts (Chikzu et al., J. Biol. Chem. 275:31444-31450, 2000), capillary endothelial cells of the blood vessels (Gonzalez et al., Pediatr Res. 39(3):375-85 1996), alveolar epithelium in the lung (Li et al., Am. J. Lung Cell Mol. Physiol. 279:L1038-1046, 2000), and spinal ganglia and sciatic nerve (Chikzu et al., ibid., 2000). Enhanced expression of FGFR1 IIIc has been identified in human breast cancer (Yoshimura et al., Clin. Immunol. Immunopathol. 89:28-34, 1998.) Therefore, in addition to diagnostic uses for zFGF11 in diseases related to expression of the cognate receptor, the molecules of the present invention can be used to detect and identify specific receptor expression levels and target molecules to tissues where the receptor is expressed.

Assays measuring differentiation include, for example, measuring cell-surface markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989; all incorporated herein by reference).

In vivo assays for evaluating neogenesis or hyperplasia include cellular proliferation assays (Stern et al., Proc. Natl. Acad. Sci. 87:6808-6812, 1990, Lok et al., Nature 369:565-568, 1994), stimulation of the proliferation of neuronal and glial progenitors isolated from the septum and striatum (Palmer et al., Mol. Cell. Neurosci. 6:474-486, 1995), and stimulation of differentiation of neurons from neural crest progenitors (Vaisman et al., Development 115:1059-1069, 1992).

In vivo assays for measuring changes in bone formation rates include performing bone histology (see, Recker, R., eds. Bone Histomorphometry: Techniques and Interpretation. Boca Raton: CRC Press, Inc., 1983) and quantitative computed tomography (QCT; Ferretti, J. Bone 17:353S-364S, 1995; Orphanoludakis et al., Investig. Radiol. 14:122-130, 1979 and Durand et al., Medical Physics 19:569-573, 1992). An ex vivo assay for measuring changes in bone formation would be, for example, a calavarial assay (Gowen et al., J. Immunol. 136:2478-2482, 1986).

With regard to modulating energy balance, particularly as it relates to adipocyte metabolism, proliferation and differentiation, zFGF11 polypeptides may modulate effects on metabolic reactions. Such metabolic reactions include adipogenesis, gluconeogenesis, glycogenolysis, lipogenesis, glucose uptake, protein synthesis, thermogenesis, oxygen utilization and the like. Among other methods known in the art or described herein, mammalian energy balance may be evaluated by monitoring one or more of the aforementioned metabolic functions. These metabolic functions are monitored by techniques (assays or animal models) known to one of ordinary skill in the art, as is more fully set forth below. For example, the glucoregulatory effects of insulin are predominantly exerted in the liver, skeletal muscle and adipose tissue. In skeletal muscle and adipose tissue, insulin acts to stimulate the uptake, storage and utilization of glucose.

Art-recognized methods exist for monitoring all of the metabolic functions recited above. Thus, one of ordinary skill in the art is able to evaluate zFGF11 polypeptides, fragments, fusion proteins, antibodies, agonists and antagonists for metabolic modulating functions. Exemplary modulating techniques are set forth below.

Insulin-stimulated lipogenesis, for example, may be monitored by measuring the incorporation of ¹⁴C-acetate into triglyceride (Mackall et al. J. Biol. Chem. 251:6462-6464, 1976) or triglyceride accumulation (Kletzien et al., Mol. Pharmacol. 41:393-398, 1992).

zFGF11-stimulated uptake may be evaluated, for example, in an assay for insulin-stimulated glucose transport. Primary adipocytes or NIH 3T3 L1 cells (ATCC No. CCL-92.1) are placed in DMEM containing 1 g/l glucose, 0.5 or 1.0% BSA, 20 mM Hepes, and 2 mM glutamine. After two to five hours of culture, the medium is replaced with fresh, glucose-free DMEM containing 0.5 or 1.0% BSA, 20 mM Hepes, 1 mM pyruvate, and 2 mM glutamine. Appropriate concentrations of zFGF11, insulin or IGF-1, or a dilution series of the test substance, are added, and the cells are incubated for 20-30 minutes. ³H or ¹⁴C-labeled deoxyglucose is added to ≈50 μM final concentration, and the cells are incubated for approximately 10-30 minutes. The cells are then quickly rinsed with cold buffer (e.g. PBS), then lysed with a suitable lysing agent (e.g. 1% SDS or 1 N NaOH). The cell lysate is then evaluated by counting in a scintillation counter. Cell-associated radioactivity is taken as a measure of glucose transport after subtracting non-specific binding as determined by incubating cells in the presence of cytochalasin b, an inhibitor of glucose transport. Other methods include those described by, for example, Manchester et al., Am. J. Physiol. 266 (Endocrinol. Metab. 29):E326-E333, 1994 (insulin-stimulated glucose transport).

Protein synthesis may be evaluated, for example, by comparing precipitation of ³⁵S-methionine-labeled proteins following incubation of the test cells with ³⁵S-methionine and ³⁵S-methionine and a putative modulator of protein synthesis.

Thermogenesis may be evaluated as described by B. Stanley in The Biology of Neuropeptide Y and Related Peptides, W. Colmers and C. Wahlestedt (eds.), Humana Press, Ottawa, 1993, pp. 457-509; C. Billington et al., Am. J. Physiol. 260:R321, 1991; N. Zarjevski et al., Endocrinology 133:1753, 1993; C. Billington et al., Am. J. Physiol. 266:R1765, 1994; Heller et al., Am. J. Physiol. 252(4 Pt 2): R661-7, 1987; and Heller et al., Am. J. Physiol. 245(3): R321-8, 1983. Also, metabolic rate, which may be measured by a variety of techniques, is an indirect measurement of thermogenesis.

Oxygen utilization may be evaluated as described by Heller et al., Pflugers Arch. 369(1): 55-9, 1977. This method also involved an analysis of hypothalmic temperature and metabolic heat production. Oxygen utilization and thermoregulation have also been evaluated in humans as described by Haskell et al., J. Appl. Physiol. 51(4): 948-54, 1981.

zFGF11 polypeptides can also be used to prepare antibodies that specifically bind to zFGF11 epitopes, peptides or polypeptides. Methods for preparing polyclonal and monoclonal antibodies are well known in the art (see, for example, Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982, which are incorporated herein by reference). As would be evident to one of ordinary skill in the art, polyclonal antibodies can be generated from a variety of warm-blooded animals, such as horses, cows, goats, sheep, dogs, chickens, rabbits, mice, and rats.

The immunogenicity of a zFGF11 polypeptide may be increased through the use of an adjuvant, such as alum (aluminum hydroxide) or Freund's complete or incomplete adjuvant. Polypeptides useful for immunization also include fusion polypeptides, such as fusions of zFGF11 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting only non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. Alternative techniques for generating or selecting antibodies useful herein include in vitro exposure of lymphocytes to zFGF11 protein or peptide, and selection of antibody display libraries in phage or similar vectors (for instance, through use of immobilized or labeled zFGF11 protein or peptide).

Antibodies are defined to be specifically binding if they bind to a zFGF11 polypeptide with a binding affinity (Kₐ) of 10⁶ M⁻¹ or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art (for example, by Scatchard analysis).

A variety of assays known to those skilled in the art can be utilized to detect antibodies which specifically bind to zFGF11 proteins or peptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant zFGF11 protein or peptide.

Antibodies to zFGF11 may be used for tagging cells that express zFGF11; to target another protein, small molecule or chemical to heart tissue; for isolating zFGF11 by affinity purification; for diagnostic assays for determining circulating levels of zFGF11 polypeptides; for detecting or quantitating soluble zFGF11 as marker of underlying pathology or disease; in analytical methods employing FACS; for screening expression libraries; for generating anti-idiotypic antibodies; and as neutralizing antibodies or as antagonists to block zFGF11 mediated proliferation in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications.

Molecules of the present invention can be used to identify and isolate receptors involved in neuronal or pancreatic cell proliferation. In particular, FGFR1 IIIc can be identified using molecules of the present invention. For example, proteins and peptides of the present invention can be immobilized on a column and membrane preparations run over the column (Immobilized Affinity Ligand Techniques, Hermanson et al., eds., Academic Press, San Diego, Calif., 1992, pp.195-202). Proteins and peptides can also be radiolabeled (Methods in Enzymol., vol. 182, “Guide to Protein Purification”, M. Deutscher, ed., Acad. Press, San Diego, 1990, 721-737) or photoaffinity labeled (Brunner et al., Ann. Rev. Biochem. 62:483-514, 1993 and Fedan et al., Biochem. Pharmacol. 33:1167-1180, 1984) and specific cell-surface proteins can be identified.

Antagonists will be useful for inhibiting the proliferative activities of zFGF11 molecules, in cell types such as neuronal, pancreatic, epithelial cells, keratinocytes, and prostatic cells, including fibroblasts and endothelial cells. For example, antagonists to zFGF11 will be useful for inhibitions of disorders associated with kidney epithelium, such as glomerulonephritis. Disorders associated with keratinocytes, such as psoriasis may be inhibited by zFGF11 antagonists. Genes encoding zFGF11 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the zFGF11 sequences disclosed herein to identify proteins which bind to zFGF11. These “binding proteins” which interact with zFGF11 polypeptides may be used for tagging cells; for isolating homolog polypeptides by affinity purification; they can be directly or indirectly conjugated to drugs, toxins, radionuclides and the like. These binding proteins can also be used in analytical methods such as for screening expression libraries and neutralizing activity. The binding proteins can also be used for diagnostic assays for determining circulating levels of polypeptides; for detecting or quantitating soluble polypeptides as marker of underlying pathology or disease. These binding proteins can also act as zFGF “antagonists” to block zFGF11 binding and signal transduction in vitro and in vivo. These anti-zFGF11 binding proteins would be useful for inhibiting expression of genes which result in proliferation or differentiation. Such anti-zFGF11 binding proteins can be used for treatment, for example, in neuroblastoma, glioblastoma, prostatic hypertrophy, prostatic carcinoma, pancreatic carcinoma, and spinal cord injury, alone or combination with other therapies.

The molecules of the present invention will be useful for proliferation of neuronal, prostatic and pancreatic tissue cells, such as pancreatic islets, pancreatic acinar cells, neuroectoderm, neurons of the central nervous systems, and sympathetic neurons in vitro. For example, molecules of the present invention are useful as components of defined cell culture media, and may be used alone or in combination with other cytokines and hormones to replace serum that is commonly used in cell culture. Molecules of the present invention are particularly useful in specifically promoting the growth and/or development of pancreatic islets, prostate cells (e.g., PZ-HPV-7 human prostate epithelium cells ATCC Number: CRL-2221 and rat YPEN-1 normal prostate cells, ATCC Number: CRL-2222); neuronal cells (e.g., mouse CATH.a brain neuronal cells ATCC Number: CRL-11179, human HCN-1A neuronal cells, ATCC Number: CRL-10442) in culture, and may also prove useful in the study of hyperplasia and regeneration. Other types of cells for which zFGF11 molecules will be useful for establishing and maintaining cell cultures include epithelial cells and keratinocytes. Epithelial cells can be isolated from, for example, prostate, cornea, lung, mammary or kidney tissues.

The polypeptides, nucleic acid and/or antibodies of the present invention may be used in treatment of disorders associated with diabetes mellitus, neural cell development or degeneration, amyotrophic lateral sclerosis, cerebrovascular stroke, neurophathy associated with lack of maintenance of neuronal differentiation, and congenital disorders of the nervous system or lack of neuronal development. Molecules of the present invention may also be useful for promoting angiogenesis and wound healing, for revascularization in the eye, for complications related to poor circulation such as diabetic foot ulcers, for stroke, following coronary reperfusion using pharmacologic methods and other indications where angiogenesis is of benefit, such as vascular diseases of the extremities. Molecules of the present invention may be useful for improving cardiac function, either by inducing cardiac myocyte neogenesis and/or hyperplasia, by inducing coronary collateral formation, or by inducing remodeling of necrotic myocardial area.

ZFGF11 will be useful for promoting wound healing of the epidermis. The molecules of the present invention can be used to protect and promote recovery of the epithelial cells in the gastrointestinal tract, small intestine and oral muscosa after treat with chemotherapy and/or radiation. Stimulation of lung epithelial cells lining the air space can promote recovery from lung injury and complications associated with premature birth in neonates. ZFGF11 may also modulate surfactant production in the lung epithelium. Other epithelial cells are found in prostate, cornea, mammary and kidney tissue, and the proliferation and specialized cell functions of these cells can be modulated by zFGF11.

An ischemic event is the disruption of blood flow to an organ, resulting in necrosis or infarct of the non-perfused region. Ischemia-reperfusion is the interruption of blood flow to an organ, such as the heart or brain, and subsequent restoration (often abrupt) of blood flow. While restoration of blood flow is essential to preserve functional tissue, the reperfusion itself is known to be deleterious. In fact, there is evidence that reperfusion of an ischemic area compromises endothelium-dependent vessel relaxation resulting in vasospasms, and in the heart compromised coronary vasodilation, that is not seen in an ischemic event without reperfusion (Cuevas et al., Growth Factors 15:29-40, 1997). Both ischemia and reperfusion are important contributors to tissue necrosis, such as a myocardial infarct or stroke. The molecules of the present invention will have therapeutic value to reduce damage to the tissues caused by ischemia or ischemia-reperfusion events, particularly in the heart or brain.

Other therapeutic uses for the present invention include induction of skeletal muscle neogenesis and/or hyperplasia, kidney regeneration and/or for treatment of systemic and pulmonary hypertension.

ZFGF11 induced coronary collateral development is measured in rabbits, dogs or pigs using models of chronic coronary occlusion (Landau et al., Amer. Heart J. 29:924-931, 1995; Sellke et al., Surgery 120(2):182-188, 1996 and Lazarous et al., 1996, ibid.) zFGF11 benefits for treating stroke is tested in vivo in rats utilizing bilateral carotid artery occlusion and measuring histological changes, as well as maze performance (Gage et al., Neurobiol. Aging 9:645-655, 1988). ZFGF11 efficacy in hypertension is tested in vivo utilizing spontaneously hypertensive rats (SHR) for systemic hypertension (Marche et al., Clin. Exp. Pharmacol. Physiol. Suppl. 1:S114-116, 1995).

Molecules of the present invention can be used to target the delivery of agents or drugs to the cells and/or tissues derived from the neuroectoderm, the developing central nervous systems, the developing peripheral nervous system, the developing spinal cord, prostate and pancreas. For example, the molecules of the present invention will be useful limiting expression to the neural tissue, by virtue of the tissue specific expression directed by the zFGF11 promoter. For example, neural tissue-specific expression can be achieved using a zFGF11-adenoviral discistronic construct (Rothmann et al., Gene Therapy 3:919-926, 1996). In addition, the zFGF11 polypeptides can be used to restrict other agents or drugs to neural tissue by linking zFGF11 polypeptides to another protein (Franz et al., Circ. Res. 73:629-638, 1993) by linking a first molecule that is comprised of a zFGF11 homolog polypeptide with a second agent or drug to form a chimera. Proteins, for instance antibodies, can be used to form chimeras with zFGF11 molecules of the present invention (Narula et al., J. Nucl. Cardiol. 2:26-34, 1995). Examples of agents or drugs include, but are not limited to, bioactive-polypeptides, genes, toxins, radionuclides, small molecule pharmaceuticals and the like. Linking may be direct or indirect (e.g., liposomes), and may occur by recombinant means, chemical linkage, strong non-covalent interaction and the like.

For pharmaceutical use, the proteins of the present invention are formulated for parenteral, particularly intravenous or subcutaneous, administration according to conventional methods. Intravenous administration will be by bolus injection or infusion over a typical period of one to several hours. In general, pharmaceutical formulations will include a zFGF11 protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington's Pharmaceutical Sciences, Gennaro, ed., Mack Publishing Co., Easton Pa., 1990, which is incorporated herein by reference. Therapeutic doses will generally be in the range of 0.1 to 100 μg/kg of patient weight per day, preferably 0.5-20 μg/kg per day, with the exact dose determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years. In general, a therapeutically effective amount of zFGF11 is an amount sufficient to produce a clinically significant change in proliferation, or increases in specific cell types associated with mesenchymal stem cells and progenitors.

ZFGF11 polypeptides can also be used to teach analytical skills such as mass spectrometry, circular dichroism, to determine conformation, especially of the four alpha helices, x-ray crystallography to determine the three-dimensional structure in atomic detail, nuclear magnetic resonance spectroscopy to reveal the structure of proteins in solution. For example, a kit containing the ZFGF11 can be given to the student to analyze. Since the amino acid sequence would be known by the instructor, the protein can be given to the student as a test to determine the skills or develop the skills of the student, the instructor would then know whether or not the student has correctly analyzed the polypeptide. Since every polypeptide is unique, the educational utility of ZFGF11 would be unique unto itself.

The antibodies which bind specifically to ZFGF11 can be used as a teaching aid to instruct students how to prepare affinity chromatography columns to purify ZFGF11, cloning and sequencing the polynucleotide that encodes an antibody and thus as a practicum for teaching a student how to design humanized antibodies. The ZFGF11 gene, polypeptide, or antibody would then be packaged by reagent companies and sold to educational institutions so that the students gain skill in art of molecular biology. Because each gene and protein is unique, each gene and protein creates unique challenges and learning experiences for students in a lab practicum. Such educational kits containing the ZFGF11 gene, polypeptide, or antibody are considered within the scope of the present invention. The invention is further illustrated by the following non-limiting examples.

In summary, the present invention includes, but is not limited to the following embodiments. The present invention provides isolated polypeptides that comprise a sequence of amino acid residues that is at least 95% identical to the sequence shown in SEQ ID NO: 2 from amino acid residue 28 to amino acid residue 208. In another embodiment, the polypeptides of the present invention further comprise a Cysteine at position 113, a Phenylaline at position 115, and a Glutamic Acid at position 117 of SEQ ID NO: 2. In further embodiments, the polypeptides of the present invention will provide polypeptides that have a Leucine at position 60, Valine at position 68, a Leucine at positions 80 and 82, Isoleucine at position 92, Leucine at position 101, Leucine at position 109, Cysteine at position 113, Phenylaline at position 115, Glutamic Acid at position 117, Phenylaline at position 122, and Tyrosine at position 134 of SEQ ID NO: 2. In another aspect, the present invention provides isolated polypeptides that comprise at least 15 contiguous amino acid residues of the sequence shown in SEQ ID NO: 2.

The present invention also provides expression vectors that comprise a transcription promoter, a DNA segment that encodes for a polypeptide described herein, and transcriptional terminator. A cultured cells expressing the polypeptide by means of the expression vectors described herein, as well as the methods for making the polypeptide are included. The present invention provides antibodies that bind to the polypeptides described herein, and the proteins that are comprised of those polypeptides.

In other aspects, the present invention provides fusion proteins that comprise at least two polypeptides wherein at least one of the polypeptides comprises a sequence of amino acid residues as shown in SEQ ID NO: 2 from residue 28 to residue 208.

In other aspects, the present invention provides isolated polynucleotide molecules that comprise a sequence of nucleotides that encode for a polypeptide as shown in SEQ ID NO: 2 from amino acid residue 28 to amino acid residue 208. In another embodiment, the present invention provides polynucleotide molecules that comprise a nucleotide sequence as shown in SEQ ID NO: 1 from nucleotide 231 to nucleotide 776, or degenerate sequences as shown in SEQ ID NO: 3 from nucleotide 82 to nucleotide 624. A further aspect of the present invention provides polynucleotide molecules that comprise a nucleotide sequence as shown in SEQ ID NO: 1 from nucleotide 150 to nucleotide 776.

Another aspect of the present invention provides methods for stimulating the proliferation of cells of the mesenchymal lineage which comprise culturing the mesenchymal stem cells or mesenchymal progenitor cells in the presence of zFGF11 polypeptide as described herein, in an amount sufficient to increase the number of mesenchymal cells as compared to cells that are grown in the absence of zFGF11.

Another aspect of the present invention provides methods for detecting the presence of zFGF11 in biological sample comprising the steps of contacting the biological sample with an antibody or an antibody fragment of claim 11, wherein the contacting is performed under conditions that allow the binding of the antibody or antibody fragment to the biological sample, and detecting any of the bound antibody or bound antibody fragment. In other aspects, instead of antibody or antibody fragments, a soluble form of FGFR1 IIIc will be used.

The present invention will also provide methods for detecting the presence of FGFR1 IIIc in biological sample comprising the steps of contacting the biological sample with zFGF11 polypeptide as described herein or polypeptide fragment as described herein, wherein the contacting is performed under conditions that allow the binding of the polypeptide or polypeptide fragment to the biological sample, and detecting any of the bound polypeptide or bound polypeptide fragment.

Methods for stimulating the proliferation and/or differentiation of cells of the osteoblastic lineage are provided as well, and will comprise culturing osteoblast progenitors or osteoblasts in the presence of zFGF11 polypeptides in an amount sufficient to increase the number of osteoblastic lineage cells as compared to osteoblastic lineage cells not grown in the presence of zFGF11 polypeptide.

The invention is further illustrated by the following non-limiting examples.

### EXAMPLES

### Example 1

Homologous recombination in yeast is used to create expression plasmids containing the polynucleotide encoding zFGF11 for expression in mammalian cells. To construct the zFGF11/pCZF199 expression vectors the following DNA fragments are transformed into S. cerevisiae: Sna BI digested pCZR199 as an acceptor vector, the zFGF11 EcoRI restriction fragment, and two, double stranded linker segments. The expression vector, pCZR199, has yeast replication elements, (CEN, ARS), the selectable marker, URA3, E. coli replication elements (e.g., AMP^{R} and ori), a blunt-ended cloning site, Sna BI, and adds either a N-terminal or C-terminal Glu-Glu tag (SEQ ID NO: 6). The vectors are used to create zFGF11 polypeptides having either end of the expressed protein Glu-Glu tagged. The double stranded linker segments are prepared using PCR. The linkers served to join the vector to the insert fragments at both the 5′ and 3′ ends. Two sets of linkers are prepared. One set of linkers joins the insert to a vector placing the Glu-Glu tag (SEQ ID NO: 6) on the 5′ end of the insert sequence using a linker. The second set of linkers is used to join the zFGF11 insert into a vector placing a 3′ Glu-Glu tag (SEQ ID NO: 6). A third set of linkers is used to join the zFGF11 insert into the vector, resulting in an untagged constructs The 5′ linker is same as the linked used for the C-terminally Glu-Glu tagged zFGF11. The 3′ linker is the same as the linker used for the N-terminally Glu-Glu tagged zFGF11. The oligonucleotides are joined using standard PCR reaction conditions and heated to 94° C. for 1.5 minutes followed by 10 cycles at 94° C. for 30 seconds, 50° C. for 1 minute and 72° C. for 1 minute, then a 10 minute extension at 72° C.

The DNA fragments are added to 100 μl competent yeast (Genetic strain SF838-9Dα, Roffman et al., EMBO J. 8:2057-65, 1989) and electroporated. The yeast cells are immediately diluted in 600 μl 1.2 M sorbitol and plated on Ura D plates and incubated at 30° C. for 48 hours. Ura⁺ colonies are selected from both the N-terminally-tagged and C-terminally-tagged zFGF11 proteins and the DNA from the resulting yeast colonies is extracted and transformed into E. coli. Individual clones harboring the correct expression construct are identified by restriction digests. DNA sequencing confirms that the desired sequences has been enjoined with one another.

Large scale plasmid DNA is isolated from one or more correct clones from both the N- and C-terminally tagged zFGF11 sequences, the expression cassette liberated from the vector and transformed into yeast or E. coli for large scale protein production.

### Example 2

The procedure described below is used for protein expressed in conditioned media of E. coli, Pichia methanolica, and chinese hamster ovary cells (CHO). For zFGF11 expressed in E. coli and Pichia, however, the media is not concentrated before application to the AF Heparin 650 m affinity column. Unless otherwise noted, all operations are carried out at 4° C. A total of 25 liters of conditioned media from CHO cells is sequentially sterile filtered through a 4 inch, 0.2 mM Millipore (Bedford, Mass.) OptiCap capsule filter and a 0.2 mM Gelman (Ann Arbor, Mich.) Supercap 50. The material is then concentrated to about 1.3 liters using a Millipore ProFlux A30 tangential flow concentrator fitted with a 3000 kDa cutoff Amicon (Bedford, Mass.) S10Y3 membrane. The concentrated material is again sterile-filtered with the Gelman filter as described above. A mixture of protease inhibitors is added to the concentrated conditioned media to final concentrations of 2.5 mM ethylenediaminetetraacetic acid (EDTA, Sigma Chemical Co. St. Louis, Mo.), 0.001 mM leupeptin (Boehringer-Mannheim, Indianapolis, Ind.), 0.001 mM pepstatin (Boehringer-Mannheim) and 0.4 mM Pefabloc (Boehringer-Mannheim).

The concentrated conditioned media is applied to a 5.0×15.0 cm AF Heparin 650 m (TosoHaas, Montgomeryville, Pa.) column equilibrated in 0.25M NaCl, 50 mM sodium phosphate, pH 7.2 at a flow rate of 5 ml/min using a BioCad Sprint HPLC (PerSeptive BioSystems, Framingham, Mass.). Two-ml fractions are collected and the absorbance at 280 nM is monitored. After sample application, the column is washed with 10 column volumes of loading buffer and when the absorbance of the effluent is less than that 0.05, the column is eluted with a three column volume gradient from 0.25 M to 2.0 M NaCl in 50 mM sodium phosphate, pH 7.2. The fractions containing zFGF11 are identified by SDS-PAGE and western blotting with anti-zFGF11 antibodies.

Fractions containing zFGF11 are pooled together and diluted ten-fold into 50 mM sodium phosphate pH 7.5 and the material is applied to a 1.5×20.0 cm Poros HS cation exchange column equilibrated in 50 mM phosphate pH 7.5 using the BioCad Sprint as described above. After sample application, the column is washed with 10 column volumes of loading buffer and when the absorbance of the effluent is less than that 0.05, the column is eluted with a 40.0 column volume gradient from 0.0 M to 2.0 M NaCl in 50 mM sodium phosphate, pH 7.5. Fractions are collected as described above and those containing zFGF11 will be identified by SDS-PAGE and Western blotting, pooled together and concentrated using an Amicon stirred cell fitted with a YM-10 membrane.

The concentrated material is then be applied to a 3.5×100 cm Sephacryl-S100 gel filtration column equilibrated in 1.0 M NaCl, 0.01 M EDTA and 0.05 M sodium phosphate, pH 7.2. Fractions are analyzed by SDS-PAGE and Western blotting with anti-zFGF11 antibodies as described above. Fractions containing pure zFGF11 are pooled together and samples are taken for amino acid analysis and N-terminal sequencing. The remainder of the sample is aliquoted, and stored at −80° C.

### Example 3

Purification of zFGF11

E. coli fermentation medium is obtained from a strain expressing zFGF11 as a Maltose Binding protein fusion. The MBPzFGF11 fusion is solubilized during sonication or French press rupture, using a buffer containing 20 mM Hepes, 0.4 M Nacl, 0.01 M EDTA, 10 mM DTT, at pH 7.4. The extraction buffer also includes 5 μg/ml quantities of Pepstatin, Leupeptin, Aprotinin, Bestatin. Phenyl methyl sulfonylfluoride (PMSF) is also included at a final concentration of 0.5 mM.

The extract is spun at 18,000×g for 30 minutes at 4° C. The resulting supernatent is processed on an Amylose resin (Pharmacia LKB Biotechnology, Piscataway, N.J.) which binds the MBP domain of the fusion. Upon washing the column, the bound MBPzFGF11 fusion is eluted in the same buffer as extraction buffer without DTT and protease inhibitors but containing 10 mM Maltose.

The eluted pool of MBPzFGF11 is treated with 1:100 (w/w) Bovine thrombin to MBPzFGF11 fusion. The cleavage reaction is allowed to proceed for 6 to 8 hours at room temperature, after which the reaction mixture is passed over a bed of Benzamidine sepharose (Pharmacia LKB Biotechnology, Piscataway, N.J.) to remove the thrombin, using the same elution buffer as described above for Amylose affinity chromatography.

The passed fraction, containing the cleaved product zFGF11 and free MBP domain are applied to a Toso Haas Heparin affinity matrix (Toso Haas, Montgomeryville, Pa.) equilibrated in 0.5 M NaCl, 20 mM Hepes, 0.01 M EDTA at pH 7.4. The MBP and zFGF11 both bound to heparin under these conditions. The bound proteins are eluted with a 2 to 3 column volume gradient formed between 0.5M NaCl and 2.0 M NaCl in column buffer.

### Example 4

For construction of adenovirus vectors, the protein coding region of human zFGF11 is amplified by PCR using primers that add PmeI and AscI restriction sites at the 5′ and 3′ termini respectively. Amplification is performed with a full-length zFGF11 cDNA template in a PCR reaction as follows: one cycle at 95° C. for 5 minutes; followed by 15 cycles at 95° C. for 1 min., 61° C. for 1 min., and 72° C. for 1.5 m followed by 72° C. for 7 min.; followed by a 4° C. soak. The PCR reaction product is loaded onto a 1.2% low-melting-temperature agarose gel in TAE buffer (0.04 M Tris-acetate, 0.001 M EDTA). The zFGF11 PCR product is excised from the gel and purified using a commercially available kit comprising a silica gel mambrane spin column (QIAquick® PCR Purification Kit and gel cleanup kit; Qiagen, Inc.) as per kit instructions. The PCR product is then digested with PmeI and AscI, phenol/chloroform extracted, EtOH precipitated, and rehydrated in 20 ml TE (Tris/EDTA pH 8). The zFGF11 fragment is then ligated into the PmeI-AscI sites of the transgenic vector pTG12-8 and transformed into E. coli DH10B™ competent cells by electroporation. Vector pTG12-8 was derived from p2999B4 (Palmiter et al., Mol. Cell Biol. 13:5266-5275, 1993) by insertion of a rat insulin II intron (ca. 200 bp) and polylinker (Fse I/Pme I/Asc I) into the Nru I site. The vector comprises a mouse metallothionein (MT-1) promoter (ca. 750 bp) and human growth hormone (hGH) untranslated region and polyadenylation signal (ca. 650 bp) flanked by 10 kb of MT-1 5′ flanking sequence and 7 kb of MT-1 3′ flanking sequence. The cDNA is inserted between the insulin II and hGH sequences. Clones containing zFGF11 are identified by plasmid DNA miniprep followed by digestion with PmeI and AscI. A positive clone is sequenced to insure that there were no deletions or other anomalies in the construct.

DNA is prepared using a commercially available kit (Maxi Kit, Qiagen, Inc.), and the zFGF11 cDNA is released from the pTG12-8 vector using PmeI and AscI enzymes. The cDNA is isolated on a 1% low melting temperature agarose gel and excised from the gel. The gel slice is melted at 70?C., and the DNA is extracted twice with an equal volume of Tris-buffered phenol, precipitated with EtOH, and resuspended in 10 μl H₂O.

The zFGF11 cDNA is cloned into the EcoRV-AscI sites of a modified pAdTrack-CMV (He, T-C. et al., Proc. Natl. Acad. Sci. USA 95:2509-2514, 1998). This construct contains the green fluorescent protein (GFP) marker gene. The CMV promoter driving GFP expression is replaced with the SV40 promoter, and the SV40 polyadenylation signal is replaced with the human growth hormone polyadenylation signal. In addition, the native polylinker is replaced with FseI, EcoRV, and AscI sites. This modified form of pAdTrack-CMV is named pZyTrack. Ligation is performed using a commercially available DNA ligation and screening kit (Fast-Link® kit; Epicentre Technologies, Madison, Wis.). Clones containing zFGF11 are identified by digestion of mini prep DNA with FseI and AscI. In order to linearize the plasmid, approximately 5 μg of the resulting pZyTrack zFGF11 plasmid is digested with PmeI. Approximately 1 μg of the linearized plasmid is cotransformed with 200 ng of supercoiled pAdEasy (He et al., ibid.) into E. coli BJ5183 cells (He et al., ibid.). The co-transformation is done using a Bio-Rad Gene Pulser at 2.5 kV, 200 ohms and 25 μFa. The entire co-transformation mixture is plated on 4 LB plates containing 25 μg/ml kanamycin. The smallest colonies are picked and expanded in LB/kanamycin, and recombinant adenovirus DNA is identified by standard DNA miniprep procedures. The recombinant adenovirus miniprep DNA is transformed into E. coli DH10B™ competent cells, and DNA is prepared using a Maxi Kit (Qiagen, Inc.) aaccording to kit instructions.

Approximately 5 μg of recombinant adenoviral DNA is digested with PacI enzyme (New England Biolabs) for 3 hours at 37° C. in a reaction volume of 100 μl containing 20-30U of PacI. The digested DNA is extracted twice with an equal volume of phenol/chloroform and precipitated with ethanol. The DNA pellet is resuspended in 10 μl distilled water. A T25 flask of QBI-293A cells (Quantum Biotechnologies, Inc. Montreal, Qc. Canada), inoculated the day before and grown to 60-70% confluence, is transfected with the PacI digested DNA. The PacI-digested DNA is diluted up to a total volume of 50 μl with sterile HBS (150 mM NaCl, 20 mM HEPES). In a separate tube, 20 μl of 1 mg/ml N-[1-(2,3-Dioleoyloxy)propyl]-N,N,N-trimethyl-ammonium salts (DOTAP) (Boehringer Mannheim, Indianapolis, Ind.) is diluted to a total volume of 100 μl with HBS. The DNA is added to the DOTAP, mixed gently by pipeting up and down, and left at room temperature for 15 minutes. The media is removed from the 293A cells and washed with 5 ml serum-free minimum essential medium (MEM) alpha containing 1 mM sodium pyruvate, 0.1 mM MEM non-essential amino acids, and 25 mM HEPES buffer (reagents obtained from Life Technologies, Gaithersburg, Md.). 5 ml of serum-free MEM is added to the 293A cells and held at 37° C. The DNA/lipid mixture is added drop-wise to the T25 flask of 293A cells, mixed gently, and incubated at 37° C. for 4 hours. After 4 hours the media containing the DNA/lipid mixture is aspirated off and replaced with 5 ml complete MEM containing 5% fetal bovine serum. The transfected cells are monitored for GFP expression and formation of foci (viral plaques).

Seven days after transfection of 293A cells with the recombinant adenoviral DNA, the cells express the GFP protein and start to form foci (viral “plaques”). The crude viral lysate is collected using a cell scraper to collect all of the 293A cells. The lysate is transferred to a 50-ml conical tube. To release most of the virus particles from the cells, three freeze/thaw cycles are done in a dry ice/ethanol bath and a 37° C. waterbath.

The crude lysate is amplified (Primary (1°) amplification) to obtain a working “stock” of zFGF11 rAdV lysate. Ten 10 cm plates of nearly confluent (80-90%) 293A cells are set up 20 hours previously, 200 ml of crude rAdV lysate is added to each 10-cm plate, and the cells are monitored for 48 to 72 hours for CPE (cytopathic effect) under the white light microscope and expression of GFP under the fluorescent microscope. When all of the 293A cells show CPE, this stock lysate is collected and freeze/thaw cycles performed as described above.

A secondary (2°) amplification of zFGF11 rAdV is then performed. Twenty 15-cm tissue culture dishes of 293A cells are prepared so that the cells are 80-90% confluent. All but 20 ml of 5% MEM media is removed, and each dish is inoculated with 300-500 ml of the 1° amplified rAdv lysate. After 48 hours the 293A cells are lysed from virus production, the lysate is collected into 250-ml polypropylene centrifuge bottles, and the rAdV is purified.

NP-40 detergent is added to a final concentration of 0.5% to the bottles of crude lysate in order to lyse all cells. Bottles are placed on a rotating platform for 10 minutes agitating as fast as possible without the bottles falling over. The debris is pelleted by centrifugation at 20,000×G for 15 minutes. The supernatant is transferred to 250-ml polycarbonate centrifuge bottles, and 0.5 volume of 20% PEG8000/2.5 M NaCl solution is added. The bottles are shaken overnight on ice. The bottles are centrifuged at 20,000×G for 15 minutes, and the supernatant is discarded into a bleach solution. Using a sterile cell scraper, the white, virus/PEG precipitate from 2 bottles is resuspended in 2.5 ml PBS. The resulting virus solution is placed in 2-ml microcentrifuge tubes and centrifuged at 14,000×G in the microcentrifuge for 10 minutes to remove any additional cell debris. The supernatant from the 2-ml microcentrifuge tubes is transferred into a 15-ml polypropylene snapcap tube and adjusted to a density of 1.34 g/ml with CsCl. The solution is transferred to 3.2-ml, polycarbonate, thick-walled centrifuge tubes and spun at 348,000×G for 3-4 hours at 25?C. The virus forms a white band. Using wide-bore pipette tips, the virus band is collected.

A commercially available ion-exchange columns (e.g., PD-10 columns prepacked with Sephadex® G-25M; Pharmacia Biotech, Piscataway, N.J.) is used to desalt the virus preparation. The column is equilibrated with 20 ml of PBS. The virus is loaded and allowed to run into the column. 5 ml of PBS is added to the column, and fractions of 8-10 drops are collected. The optical densities of 1:50 dilutions of each fraction are determined at 260 nm on a spectrophotometer. Peak fractions are pooled, and the optical density (OD) of a 1:25 dilution is determined. OD is converted to virus concentration using the formula: (OD at 260 nm)(25)(1.1×10¹²)=virions/ml.

To store the virus, glycerol is added to the purified virus to a final concentration of 15%, mixed gently but effectively, and stored in aliquots at −80° C.

A protocol developed by Quantum Biotechnologies, Inc. (Montreal, Canada) is followed to measure recombinant virus infectivity. Briefly, two 96-well tissue culture plates are seeded with 1×10⁴ 293A cells per well in MEM containing 2% fetal bovine serum for each recombinant virus to be assayed. After 24 hours 10-fold dilutions of each virus from 1×10⁻² to 1×10⁻¹⁴ are made in MEM containing 2% fetal bovine serum. 100 μl of each dilution is placed in each of 20 wells. After 5 days at 37° C., wells are read either positive or negative for CPE, and a value for “Plaque Forming Units/ml” (PFU) is calculated.

### Example 5

A panel of cDNAs from human tissues is screened for zFGF11 expression using PCR. The panel is made in-house and contained 94 marathon cDNA and cDNA samples from various normal and cancerous human tissues and cell lines is shown in Table 4, below. The cDNAs come from in-house libraries or marathon cDNAs from in-house RNA preps, Clontech RNA, or Invitrogen RNA. The marathon cDNAs are made using the marathon-Ready™ kit (Clontech, Palo Alto, Calif.) and QC tested with clathrin primers, and then diluted based on the intensity of the clathrin band. To assure quality of the panel samples, three tests for quality control (QC) are run: (1) To assess the RNA quality used for the libraries, the in-house cDNAs are tested for average insert size by PCR with vector oligos that are specific for the vector sequences for an individual cDNA library; (2) Standardization of the concentration of the cDNA in panel samples is achieved using standard PCR methods to amplify full length alpha tubulin or G3PDH cDNA using a 5′ vector oligonucleotide and 3′ alpha tubulin specific oligonucleotide primer or 3′ G3PDH specific oligo primer; and (3) a sample is sequenced to check for possible ribosomal or mitochondrial DNA contamination. The panel is set up in a 96-well format that included a human genomic DNA (Clontech, Palo Alto, Calif.) positive control sample. Each well contains approximately 0.2-100 pg/μl of cDNA. The PCR reactions are set up using appropriate oligonucleotides, TaKaRa Ex Taq™ (TAKARA Shuzo Co LTD, Biomedicals Group, Japan), and Rediload dye (Research Genetics, Inc., Huntsville, Ala.). The typical amplification is carried out as follows: 1 cycle at 94° C. for 2 minutes, 35 cycles of 94° C. for 30 seconds 66.3° C. for 30 seconds and 72° C. for 30 seconds, followed by 1 cycle at 72° C. for 5 minutes. About 10 μl of the PCR reaction product is subjected to standard Agarose gel electrophoresis using a 4% agarose gel. The correct predicted DNA fragment size is observed in: (1) normal tissues from fetal liver, thyroid, testis, B cells, lung, and prostate; and (2) cancerous tissues from lung, liver, ovary, rectum and uterus.

Table 4

| | # | | # |
|---|---|---|---|
| Tissue/Cell line | samples | Tissue/Cell line | samples |
| Adrenal gland | 1 | Bone marrow | 3 |
| Bladder | 1 | Fetal brain | 3 |
| Bone Marrow | 1 | Islet | 2 |
| Brain | 1 | Prostate | 3 |
| Cervix | 1 | RPMI #1788 (ATCC # CCL-156) | 2 |
| Colon | 1 | Testis | 4 |
| Fetal brain | 1 | Thyroid | 2 |
| Fetal heart | 1 | WI38 (ATCC # CCL-75 | 2 |
| Fetal kidney | 1 | ARIP (ATCC # CRL-1674-rat) | 1 |
| Fetal liver | 1 | HaCat-human keratinocytes | 1 |
| Fetal lung | 1 | HPV (ATCC # CRL-2221) | 1 |
| Fetal muscle | 1 | Adrenal gland | 1 |
| Fetal skin | 1 | Prostate SM | 2 |
| Heart | 2 | CD3+ selected PBMC's | 1 |
| | | Ionomycin + PMA stimulated | |
| K562 (ATCC # | 1 | HPVS (ATCC # CRL-2221)- | 1 |
| CCL-243) | | selected | |
| Kidney | 1 | Heart | 1 |
| Liver | 1 | Pituitary | 1 |
| Lung | 1 | Placenta | 2 |
| Lymph node | 1 | Salivary gland | 1 |
| Melanoma | 1 | HL60 (ATCC # CCL-240) | 3 |
| Pancreas | 1 | Platelet | 1 |
| Pituitary | 1 | HBL-100 | 1 |
| Placenta | 1 | Renal mesangial | 1 |
| Prostate | 1 | T-cell | 1 |
| Rectum | 1 | Neutrophil | 1 |
| Salivary Gland | 1 | MPC | 1 |
| Skeletal muscle | 1 | Hut-102 (ATCC # TIB-162) | 1 |
| Small intestine | 1 | Endothelial | 1 |
| Spinal cord | 1 | HepG2 (ATCC # HB-8065) | 1 |
| Spleen | 1 | Fibroblast | 1 |
| Stomach | 1 | E. Histo | 1 |
| Testis | 2 | | |
| Thymus | 1 | | |
| Thyroid | 1 | | |
| Trachea | 1 | | |
| Uterus | 1 | | |
| Esophagus tumor | 1 | | |
| Gastric tumor | 1 | | |
| Kidney tumor | 1 | | |
| Liver tumor | 1 | | |
| Lung tumor | 1 | | |
| Ovarian tumor | 1 | | |
| Rectal tumor | 1 | | |
| Uterus tumor | 1 | | |

### Example 6

Binding assays are performed to identify any known FGF receptors that bind zFGF11. BaF3 cell lines are engineered to express the receptors shown in Table 5. BaF3 cells are murine pre-B cells that are dependent on IL-3 for growth, and when engineered to express FGF receptors, require either IL-3 or the appropriate ligand for the expressed FGF receptor type.

TABLE 5

| Cell Line | Type of FGF Receptor |
|---|---|
| FR1C-11 | FGFR1 IIIc |
| FR1B-5 | FGFR1 IIIb |
| FR2C-2 | FGFR2 IIIc |
| FR2B-7 | FGFR2 IIIc |
| FR31C-4 | FGFR3 IIIc outside/FGFR 1 inside |
| | (chimera) |
| FR31BQ | FGFR3 IIIb outside/FGFR 1 inside |
| | (chimera) |

BaF3 cell lines are cultured in media containing RPMI 1640 (Life Technologies, Rockville, Md.) and 10% fetal calf serum (Hyclone, Logan, Utah) with 1 ng/ml IL-3 (R&D Systems, ). One hundred microliters of assay medium is added to each well of a 96-well microtiter dish as shown in Table 6.

TABLE 6

| conditioned medium | concentration |
|---|---|
| IL-3 (control) | 20 pg/ml |
| FGF-1 (control) | 200 ng/ml |
| FGF-2 (control) | 200 ng/ml |
| FGF-18 (zFGF5; control) | 200 ng/ml |
| adenoviral vector | concentrated 10X |
| without insert | |
| adenoviral zFGF11 | concentrated 10X |
| adenoviral zFGF12 | concentrated 10X |

1×10⁴ cells/well are added, and the dishes are grown for three days. After incubation, Alamar Blue dye (a metabolic dye that is incorporated into proliferating cells) is added to each well, and the cells are grown overnight. The next day, cell proliferation is determined using OD₅₄₄₋₅₉₀ as an index. Table 7 contains the data for each well, with Growth Medium being unconditioned medium, adenovirus vector medium being medium conditioned with cells transfected with adenovirus vector without any insert.

The results demonstrate that conditioned medium derived from cells transfected with adenovirus constructs expressing zFGF11 significantly increased the proliferation of cells harboring the FGF receptor IIIc.

TABLE 7

| | | | | | | | | | | Adeno- | |
|---|---|---|---|---|---|---|---|---|---|---|---|
| | | | | | | | | | | Virus | |
| | Growth | | | | Growth | | Growth | | Growth | Cond. | Growth |
| IL-3 | Medium | aFGF | bFGF | zFGF-5 | Medium | zFGF-11 | Medium | zFGF12 | Medium | Medium | Medium |
| 4262.554 | 1414.132 | 4422.96 | 4567.164 | 1406.092 | 1313.83 | 3912.48 | 1321.76 | 2512.691 | 1440.061 | 2219.13 | 1567.98 |
| 3876.472 | 1520.904 | 4335.224 | 5059.6 | 1467.663 | 1327.21 | 2019.82 | 1454.7 | 2184.82 | 1424.81 | 1897.804 | 1398.532 |
| 3749.65 | 1511.19 | 4109.85 | 4985.83 | 1515.071 | 1370.99 | 1761.5 | 1374.362 | 1922.49 | 1389.274 | 1557.352 | 1446.57 |
| 3459.783 | 1568.48 | 3806.39 | 5422.33 | 1412.092 | 1386.611 | 1440.18 | 1444.87 | 1710.26 | 1418.032 | 1295.31 | 1541.73 |
| 2838.412 | 1534.442 | 3458.5 | 4969.49 | 1546.13 | 1427.69 | 1319.79 | 1506.82 | 1436.95 | 1462.354 | 1264.74 | 1484.574 |
| 2369.031 | 1551.114 | 3533.221 | 4631.064 | 1567.492 | 1509.13 | 1355.36 | 1481.573 | 1417.801 | 1453.54 | 1257.66 | 1568.77 |
| 2168.751 | 1549.292 | 3648.13 | 4433.09 | 1534.01 | 1574.54 | 1354.344 | 1703.774 | 1311.96 | 1439.34 | 1338.153 | 1654.77 |
| 1846.33 | 1555.232 | 3891.954 | 4596.683 | 1629.28 | 1496.243 | 1345.71 | 1580.522 | 1371.23 | 1560.302 | 1312.5 | 1536.28 |

### Example 7

Embryonic stem (ES) cells are derived from blastocytes. ES are multipotent cells capable of differentiating in vitro via embryo-like aggregates, so-called “embryo bodies”, into derivatives of the endodermal, ectodermal and mesodermal lineage. It has been demonstrated that a variety of terminally differentiated cells can be derived from ES cells, such as skeletal muscle, smooth muscle and cardiac muscle cells, neurons, endothelial cells, hematopoietic cells, and lymphocytes etc. An ES cell differentiation system is used to screen biological activities of proteins, such as FGF that may affect proliferation and differentiation of a particular tissue or lineage.

ES cells are grown on mono layer of “feeder” cells, i.e., mouse embryo fibroblast cell line (SNL76/7; McMahon et al., Cell, 62:1153 (1991).) in standard ES culture media (DMEM-Dulbecco's modified Eagle's medium (GIBCO-BRL, Gaithersburg, Md.) with Pen/Strep, 1 mM glutamine (GIBCO BRL), 0.1 mM beta-mercapto-ethanol (Sigma), and 20% FBS (HyClone)).

Embryo bodies (EB) are prepared by trypsinizing ES cells, and resuspending in ES media. The cells are transferred to a tissue culture petri dish and incubated in tissue culture chamber for 30 minutes to selectively attach feeder cells ES cells.

ES cells are harvested, counted and resuspended at the concentration of 2.4×10³/ml in differentiation media,; i.e., ES media without beta-mercapto-ethanol. Hanging drops of 50 μl ES cells are made on the inside lid of tissue culture plates so that each drop contains 1200 ES cells. Simultaneously, adenovirus expression vector for zFGF11 (Adzyzfgf11) is introduced to infect ES cells at the concentration of 10,000 viral particles/ES cells. The parental vector (vector without zFGF11 insert) is also used to infect ES cells as a control.

After allowing ES cells to form embryo body (EB), the EB are incubated for four days, and are harvested and transferred into tissue culture plates. The EB then attach to the bottom of tissue culture plates. Cells are allowed proliferate and differentiate into variety of lineage for next twelve days. Culture media is refreshed by addition of fresh differentiation media every three days.

Eight days after attachment, differentiated cells are harvested and washed with PBS. RNA is isolated by RNAzo1 (GibcoBRL), and digested by RNAse-free DNAse to get rid of contaminating genomic DNA.

Expression of over 1000 mouse genes in treated samples are compared to controls by using Clontech Atlas™ cDNA expression array (Mouse 1.0 Nylon Array) and AtlasImage™ software (Clontech, Palo Alto, Calif.). Results demonstrated that zFGF11 induces expression of osteoblast-specific factor 2 precursor (OSF2) by 9 fold. zFGF11 also induces a large number of neuronal factors, including zinc finger protein of the cerebellum (ZIC1), Iroquois-related homeobox protein (IRX3), Hox-8, N-cadherin precursor, enabled homolog (ENAH), and kinesin family protein (KIFIA). Hematopoietic genes, such as lymphocyte activation antigen CD30 (ki-1 antigen in Hodgkin's disease), T-cell death-associated protein (TDAG51), PDGF receptor, and EPO receptor. These results indicate that zFGF11 affect bone formation, neuronal differentiation and certain hematopoietic cells.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. An isolated polypeptide comprising a sequence of amino acid residues that is at least 95% identical to the sequence as shown in SEQ ID NO:2 from residue 28 through residue 208.

2. The isolated polypeptide of claim 1 wherein the polypeptide comprises a Cys residue at position 113, a Phe residue at position 115 and a Glu residue at position 117 of SEQ ID NO:2.

3. The isolated polypeptide of claim 1 wherein the polypeptide comprises residue 60 (Leu), residue 68 (Val), residue 80 (Leu), residue 82 (Leu), residue 90 (Ile), residue 92 (Ile), residue 101 (Leu), residue 109 (Leu), residue 113 (Cys), residue 115 (Phe), residue 117 (Glu), residue 122 (Phe), and residue 134 (Tyr) of SEQ ID NO:2.

4. An isolated polypeptide comprising the amino acid sequence of SEQ ID NO:2 from residue 28 through residue 208.

5. An isolated polypeptide comprising at least 15 contiguous amino acid residues of SEQ ID NO:2.

6. An expression vector comprising the following operably linked elements:
(a) a transcription promoter;
(b) a DNA segment encoding a polypeptide according to claim 1; and
(c) a transcription terminator.

7. The expression vector of claim 6 further comprising a secretory signal sequence operably linked to the DNA segment.

8. An expression vector comprising the following operably linked elements:
(a) a transcription promoter;
(b) a DNA segment encoding a polypeptide according to claim 4; and
(c) a transcription terminator.

9. A cultured cell comprising the expression vector of claim 6.

10. A method of making a polypeptide comprising:
culturing a cell according to claim 9 under conditions wherein the DNA segment is expressed; and
recovering the polypeptide encoded by the DNA segment.

11. An antibody that specifically binds to the polypeptide of claim 1 or a protein comprising the polypeptide of claim 1.

12. An isolated polynucleotide molecule comprising a sequence of nucleotides that encode for a sequence of amino acid residues that is at least 95% identical to the sequence as shown in SEQ ID NO:2 from residue 28 through residue 208.

13. An isolated polynucleotide molecule comprising a sequence of nucleotides as shown in SEQ ID NO: 1 from nucleotide 231 to nucleotide 776 or SEQ ID NO: 3 from nucleotide 82 to nucleotide 624.

14. The isolated polynucleotide molecule of claim 13, wherein the nucleotide sequence is from nucleotide 150 to nucleotide 776 as shown in SEQ ID NO: 1.

15. A fusion protein comprising at least two polypeptides wherein at least one of the polypeptides comprises a sequence of amino acid residues as shown in SEQ ID NO: 2 from amino acid residue 28 to amino acid residue 208.

16. A method of stimulating proliferation of mesenchymal cells comprising culturing mesenchymal stem cells, progenitor cells or mesenchymal cells in the presence of zFGF11 polypeptide comprising a sequence of amino acid residues as shown in SEQ ID NO: 2 from residue 28 to residue 208, in an amount sufficient to increase the number of mesenchymal cells as compared to mesenchymal cells grown in the absence of zFGF11 polypeptide.

17. A method of detecting the presence of zFGF11 in a biological sample, comprising the steps of:
(a) contacting the biological sample with an antibody or an antibody fragment of claim 11, wherein the contacting is performed under conditions that allow the binding of the antibody or antibody fragment to the biological sample, and
(b) detecting any of the bound antibody or bound antibody fragment.

18. A method of detecting the presence of zFGF11 in a biological sample, comprising the steps of:
(a) contacting the biological sample with soluble FGFR1 IIIc, wherein the contacting is performed under conditions that allow the binding of the receptor to the biological sample, and
(b) detecting any of the bound receptor.

19. A method of detecting the presence of FGFR1 IIIc in a biological sample, comprising the steps of:
(a) contacting the biological sample with an zFGF11 polypeptide or a polypeptide fragment of claim 4, wherein the contacting is performed under conditions that allow the binding of the polypeptide or polypeptide fragment to the biological sample, and
(b) detecting any of the bound polypeptide or bound polypeptide fragment.

20. A method of stimulating proliferation of osteoblastic lineage cells comprising culturing osteoblast progenitors or osteoblasts in the presence of zFGF11 polypeptide comprising a sequence of amino acid residues as shown in SEQ ID NO: 2 from residue 28 to residue 208, in amount sufficient to increase the number of osteoblastic lineage cells as compared to osteoblastic lineage cells grown in the absence of zFGF11 polypeptide.

21. A method of forming bone by stimulating proliferation of osteoblasts comprising administering to a mammal in need thereof, a therapeutically effective amount of a zfgf11 polypeptide comprising a sequence of amino acids as shown in SEQ ID NO:2 from 28 to residue 208.

22. A method of treating a bone defect by stimulating proliferation of osteoblasts by administering to a mammal in need thereof, a therapeutically effective amount of a zfgf11 polypeptide comprising a sequence of amino acids as shown in SEQ ID NO:2 from 28 to residue 208.

23. A method of stimulating bone formation in a bone fracture by stimulating proliferation of osteoblasts comprising administering to a mammal in need thereof, a therapeutically effective amount of a zfgf11 polypeptide comprising a sequence of amino acids as shown in SEQ ID NO:2 from 28 to residue 208.

24. A method of treating osteoporosis by stimulating proliferation of osteoblasts comprising administering to a mammal in need thereof, a therapeutically effective amount of a zfgf11 polypeptide comprising a sequence of amino acids as shown in SEQ ID NO:2 from 28 to residue 208.


SEQUENCE LISTING (6 sequences)

SEQ ID NO: 1 - DNA, 882 bp, Homo sapiens
  CDS (150)...(776)
tgtcagctgaggatccagccgaaagaggagccaggcactcaggccacctgagtctactcacctggacaactggaatctggcaccaattctaaaccactcagcttctccgagctcacaccccggagatcacctgaggacccgagccattgatggactcggacgagaccgggttcgagcactcaggactgtgggtttctgtgctggctggtctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctgaagccagaggctgtttactatgacatctcctctttatttattaggttatttatcttatttatttttttatttttcttacttgagataataaagagttccagaggaga

SEQ ID NO: 2 - protein, 208 aa, Homo sapiens
MDSDETGFEHSGLWVSVLAGLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 3 - DNA, 624 bp, Artificial Sequence
  degenerate sequence
atggaywsngaygaracnggnttygarcaywsnggnytntgggtnwsngtnytngcnggnytnytnggngcntgycargcncayccnathccngaywsnwsnccnytnytncarttyggnggncargtnmgncarmgntayytntayacngaygaygcncarcaracngargcncayytngarathmgngargayggnacngtnggnggngcngcngaycarwsnccngarwsnytnytncarytnaargcnytnaarccnggngtnathcarathytnggngtnaaracnwsnmgnttyytntgycarmgnccngayggngcnytntayggnwsnytncayttygayccngargcntgywsnttymgngarytnytnytngargayggntayaaygtntaycarwsngargcncayggnytnccnytncayytnccnggnaayaarwsnccncaymgngayccngcnccnmgnggnccngcnmgnttyytnccnytnccnggnytnccnccngcnytnccngarccnccnggnathytngcnccncarccnccngaygtnggnwsnwsngayccnytnwsnatggtnggnccnwsncarggnmgnwsnccnwsntaygcnwsn

SEQ ID NO: 4 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 5 - protein, 5 aa, Artificial Sequence
  motif
CXFXE

SEQ ID NO: 6 - protein, 5 aa, Artificial Sequence
  peptide affinity tag
EYPME